(19) United States
(12) Patent Application Publication
(10) Pub. No.: US 2019/0085086 A1
(43) Pub. Date: Mar. 21, 2019

(54) SCREENING METHODS FOR IDENTIFYING AND TREATING HIV-1 INFECTED PATIENT SUB-POPULATIONS SUITABLE FOR LONG TERM ANTI-CCR5 AGENT THERAPY

(71) Applicant: CytoDyn Inc., Vancouver, WA (US)

(72) Inventors: Paul J. Maddon, Scarsdale, NY (US); Nader Z. Pourhassan, Vancouver, WA (US)

(21) Appl. No.: 16/134,658

(22) Filed: Sep. 18, 2018

Related U.S. Application Data
(60) Provisional application No. 62/560,000, filed on Sep. 18, 2017.

(51) Int. Cl. C07K 16/28; G01N 33/569

(52) U.S. Cl. CPC C07K 16/2866; G01N 33/56988; A61K 2039/505; C07K 2317/24; G01N 2333/16

(57) ABSTRACT

Certain R5 virus tropic HIV-1 subjects with viral load effectively conventionally controlled using HAART, i.e., subject having less than 50 viral copies/mL (<50 cp/mL), may be substantially more susceptible than others to effective monotherapy treatment using anti-CCR5 agents, e.g, PRO 140 mAbs. Certain HIV-1 subjects using PRO 140 monotherapy treatment may experience prolonged or unlimited time periods with actual undetectable viral loads, extremely low viral load counts ≤1 cp/mL, very low, or low levels, or at conventionally undetectable levels, during monotherapy. Increasing dose amounts of anti-CCR5 agents, e.g., PRO 140, from 350 mg to 525 mg or 700 mg, may beneficially suppress a subject's viral load count before, during, and/or maintain effective prolonged monotherapy and may shorten the period of time necessary to determine if a subject will respond positively to PRO 140 monotherapy to less than eight (8) weeks. This invention includes protocols, methods, and kits.

### BACKGROUND

### Technical Field

The present disclosure relates to identification and treatment of HIV-1-infected patient subpopulations most likely to experience prolonged viral load suppression at actual undetectable, extremely low, very low, or low levels, or at conventionally undetectable levels, during monotherapy. In one aspect, the invention involves, among other things, a single-copy assay (SCA), which can quantify HIV-1 viremia at levels down to <1 copy per milliliter (mL) of plasma, to screen potential subjects, or to measure treatment effectiveness. In another aspect, the invention involves, among other things, high-dose anti-CCR5 agent monotherapy to further ensure maximal viral load suppression of potential subjects prior to, upon initiation of, or during treatment, to better maintain viral load suppression at actual undetectable, extremely low, very low, or low levels, or at conventionally undetectable levels, during monotherapy during prolonged treatment. SCAs and high-dose anti-CCR5 agent monotherapy may, or may not, be used in combination.

### Background

Highly active antiretroviral therapy (HAART) has transformed management of HIV-1 infection and offers the potential for a normal life expectancy for many individuals with access to care. Bhaskaran K, Hamouda O, Sannes M, et al., Changes in the risk of death after HIV seroconversion compared with mortality in the general population, JAMA. 2008; 300 (1):51-9.; Antiretroviral Therapy Cohort Collaboration. Life expectancy of individuals on combination antiretroviral therapy in high-income countries: a collaborative analysis of 14 cohort studies, LANCET, 2008; 372(9635):293-9. However, even when effective in reducing plasma viremia to levels that are “undetectable” in conventional, or standard, assays, i.e., less than 50 viral copies/mL (<50 cp/mL plasma), HAART does not eradicate HIV-1, and long-term morbidities still occur. Effros R B, Fletcher C V, Gebo K, et al., Aging and infectious diseases: workshop on HIV infection and aging: what is known and future research directions, CLIN INFECT DIS. 2008; 47 (4):542-53; Weber R, Sabin C A, Friis-Moller N, et al., Liver-related deaths in persons infected with the human immunodeficiency virus: the D:A:D study, ARCH INTERN MED. 2006; 166 (15):1632-41; Mondy K, Tebas P., Cardiovascular risks of antiretroviral therapies, ANNU REV MED. 2007; 58:141-55; and Robertson K R, Smurzynski M, Parsons T D, et al., The prevalence and incidence of neurocognitive impairment in the HAART era, AIDS. 2007; 21 (14):1915-21. In addition, treatment fails to achieve or maintain optimal viral suppression in many individuals. Indeed, most HIV-1-infected patients receiving HAART with plasma HIV-1 RNA levels below the detection limits of conventional commercial assays have residual viremia measurable by more sensitive methods. Gandhi et al., The effect of intensification on low level residual viremia in HIV infected patients on antiretroviral therapy: a randomized controlled trial, PLOS MEDICINE, August 2010 vol. 7 issue 8 (“Gandhi”); Archon et al., Antiretroviral intensification and valproic acid lacks a sustained affect on residual HIV-1 viremia or resting CD4+ cell infection, PLOS MEDICINE, February 2010, vol. 5 issue 2.

In HIV-1 infection, plasma virus levels have proven to be an important indicator of viral replication, risk of disease progression, and response to therapy. Dinoso et al., Treatment intensification does not reduce residual HIV-1 viremia in patients on highly active antiretroviral therapy, PNAS, Jun. 9, 2009 vol. 106 no. 23 (“Dinoso”). Initial studies of changes in viremia in response to antiretroviral drugs demonstrated exponential reductions occurring in at least 2 distinct phases. These phases of decay have half-lives of ˜1 day and ˜14 days, reflecting the lifespan of HIV-infected CD4⁺ T lymphoblasts and that of a second, longer-living population of infected cells, respectively. Within several weeks, plasma virus levels fall to below the limit of detection of HIV-1 RNA assays approved for patient management (50 copies per mL of plasma). On the basis of these studies, there was initial optimism that HIV-1 could be eradicated with prolonged antiretroviral therapy.

However, since the initial studies, several discoveries tempered hope for eradication. The first was the identification of a long-lived latent reservoir of HIV-1 in resting CD4⁺ T cells. Latently infected cells persist in patients on HAART who have suppression of viremia to levels below the detection limit of clinical assays. With a half-life estimated at 44 months, this compartment may not be eliminated within the lifespan of most infected individuals. A second discovery was that most patients whose HIV-1 RNA levels were suppressed by HAART to <50 copies per mL were actually viremic at a low level. Novel quantitative techniques, including the SCA, can quantify HIV-1 viremia at levels down to ≤1 copy per mL, allowing a more detailed analysis of the viral decay kinetics on HAART. Studies using the SCA revealed that the initial 2-phase decline in viremia is followed by a prolonged third phase of decay occurring over months. Subsequently, there appears to be a stable fourth phase during which there is no appreciable decay. The median level of the residual viremia during this fourth phase is ˜1.5 copies per mL. See Dinoso, cited above.

Persistent or residual viremia has been a recognized problem and is a subject of ongoing study. Zheng; Dinoso; Grant et al., Switch from enfuvirtide to raltegravir in virologically suppressed HIV-1 patients: effects on the level of residual viremia and quality of life, J. CLIN. VIRAL. 2009 December; 46 (4):305-308. It has been suggested that persistent viremia might explain the observation that T-cell activation remains higher in patients who are receiving therapy and have HIV-1 RNA levels of <50 copies/mL than in uninfected individuals. This persistent immune activation may have important clinical consequences; for example, persistent T cell activation is associated with lower CD4 cell count increases in patients receiving HAART and may contribute to accelerated atherosclerosis or premature immunosenescence. See Gandhi, above.

Low-level viremia may represent ongoing replication or release of virus from long-lived cellular reservoirs, such as resting memory CD4 cells, and likely other, as yet undefined, sources. Zheng et al., Predictors of residual viremia in patients on long-term suppressive antiretroviral therapy, ANTIVIR. THER. 2013; 18(1) (“Zheng”). In one report, by Zheng, a study evaluated factors associated with residual viremia in patients on suppressive HAART who underwent screening for a raltegravir intensification trial (ACTG A5244). The screened population was HIV-1-infected adults receiving HAART for ≥12 months with pre-ART HIV-1 RNA>100,000 copies/ml and on-therapy RNA levels below detection limits of commercial assays for ≥6 months. Of 103 patients eligible for analysis, the median age was 46 years and the median duration of viral suppression was 4.8 years. Of these 103 patients, 62% had detectable viremia (>0.2 copies/ml) by single copy assay (SCA) (median 0.2 copies/ml, IQR <0.2-1.8). It was determined that younger patients had lower HIV-1 RNA levels than older individuals (r=0.27, P=0.005). Also, patients with virological suppression on HAART for 2 years or less had higher residual viremia than those with suppression for >2 years (median 2.3 versus 0.2 copies/ml; P=0.016).

It is also noted that neurocognitive disorders remain common among human immunodeficiency virus (HIV)-positive adults, perhaps owing to persistent HIV-1 RNA in cerebrospinal fluid (CSF) during antiretroviral therapy. Anderson et al., Prevalence and correlates of persistent HIV-1 RNA and cerebrospinal fluid during antiretrovirals therapy, JID 2017: 215 (1 January) (“Anderson”). That is, human immunodeficiency virus (HIV)-associated neurocognitive disorder (HAND) is common, with a prevalence ranging from 30% to 70% among HIV-infected adults, including those taking HAART. Several explanations may account for this, including advancing age, longer duration of exposure to HIV, comorbid conditions, and more-advanced immune suppression. Another, nonexclusive explanation for high HAND prevalence among treated individuals is incomplete effectiveness or toxicity of HAART in the central nervous system (CNS).

It is reported that HIV-1 enters the CNS soon after infection and can be protected in this compartment from immune and drug pressure (see Anderson, cited above). Autopsy and neuroimaging studies have identified that HIV-1 can localize in the basal ganglia and hippocampus, even during the first weeks of infection. Potent HAART can reduce the HIV-1 level in blood and cerebrospinal fluid (CSF) below the quantification limit of commercially available assays, but HIV-1 might continue to replicate at low levels, increasing the risk for viral compartmentalization in the CNS. Persistent low-level HIV-1 replication could also lead to glial activation and neuronal injury. Published reports have identified that low-level HIV-1 is present in CSF in up to 28% of adults taking HAART but have not found associations with estimated HAART drug distribution into the CNS or neurocognitive outcomes.

A study by Anderson used an SCA to measure HIV-1 RNA levels in CSF and plasma specimens from 220 HIV-positive adults who were taking suppressive HAART. HIV-1 RNA was detected in 42.3% of CSF and 65.2% of plasma samples. Correlates of higher CSF HIV-1 RNA levels included higher nadir and current CD4+ T-cell counts, a plasma HIV-1 RNA level of ≥1 copy/mL, and a lower central nervous system penetration-effectiveness score (model P<0.001). Worse neurocognitive performance was associated with discordance in HIV-1 RNA detection between plasma and CSF, lower overall CSF HIV-1 RNA level, and longer HAART duration, among others (model P<0.001). In the longitudinal subgroup, CSF HIV-1 RNA persisted in most participants (69%) over 7 months. It was concluded that low-level HIV-1 RNA in CSF is common during suppressive HAART and is associated with low-level HIV-1 RNA in blood, better immune status, and lower HAART drug distribution into CSF. The association between HIV-1 RNA discordance and HIV-associated neurocognitive disorder (HAND) may reflect compartmentalization. The relationship between HAND, lower HIV-1 RNA levels in CSF, and lower CD4+T-cell counts may reflect disturbances in the immune response to HIV-1 in the CNS.

Substantial progress has been made over the past two decades in the development of effective and well tolerated combination antiretroviral regimens. Most HIV-1 infected persons who initiate antiretroviral therapy at early stages in the disease process and who are fully adherent to their antiretroviral regimens can anticipate life expectancies that are measured in decades. Although these advances have revolutionized antiretroviral therapy for most HIV-1 infected patients, contemporary lifelong daily adherence to treatment regimens remains challenging for a significant subset of patients. A number of studies have been conducted to evaluate the possibility of treatment simplification following control of viral replication with an induction regimen. Arribas, et al., Lopinavir/ritonavir as single-drug therapy for maintenance of HIV-1 viral suppression: 48-week results of a randomized, controlled, open-label, proof-of-concept pilot clinical trial (OK Study), J ACQUIR IMMUNE DEFIC SYNDR., Vol. 40, pp. 280-287 (2005); Pulido F et al., Lopinavir-ritonavir monotherapy versus lopinavir-ritonavir and two nucleosides for maintenance therapy of HIV, AIDS, Vol. 22., pp. F1-9 (2008); Moltó J. et al., Lopinavir/ritonavir monotherapy as a simplification strategy in routine clinical practice. J ANTIMICROB CHEMOTHER., Vol. 60, pp. 436-439 (2007); Cameron D W et al., A 96-week comparison of lopinavir-ritonavir combination therapy followed by lopinavir-ritonavir monotherapy versus efavirenz combination therapy, INFECT DIS., Vol. 198, pp. 234-240 (2008); Nunes E P et al., Monotherapy with Lopinavir/Ritonavir as maintenance after HIV-1 viral suppression: results of a 96-week randomized, controlled, open-label, pilot trial (KalMo study), HIV CLIN TRIALS., Vol. 10. pp. 368-374 (2009); Meynard J L et al., Lopinavir/ritonavirmonotherapy versus current treatment continuation for maintenance therapy of HIV-linfection: the KALESOLO trial, J ANTIMICROB CHEMOTHER., Vol. 65., pp. 2436-2444 (2010); Katlama C et al., Efficacy of darunavir/ritonavir maintenance monotherapy in patients with HIV-1 viral suppression: a randomized open-label, noninferiority trial MONOI-ANRS 136, AIDS, Vol. 24, pp. 2365-2374 (2010); Gutmann C et al., Randomized controlled study demonstrating failure of LPV/r monotherapy in HIV: the role of compartment and CD4-nadir. AIDS, Vol. 24, pp. 2347-2354 (2010); Cahn P et al., Pilot, randomized study assessing safety, tolerability and efficacy of simplified LPV/r maintenance therapy in HIV patients on the 1st PI-based regimen, PLoS ONE, Vol. 6, p. e23726 (2011); and Guiguet M et al., Boosted protease inhibitor monotherapy as a maintenance strategy: an observational study, AIDS, Vol. 26, pp. 2345-50 (2012). Most of these simplification trials have involved the substitution of a boosted HIV-1 protease inhibitor such as lopinavir or darunavir for an effective combination regimen. Although the strategy has been successful in a substantial fraction of those who undergo regimen simplification, the overall body of evidence suggests that boosted protease inhibitor maintenance therapy is generally less effective than maintenance on a three drug regimen. Calza L Manfredi R, Protease inhibitor monotherapy as maintenance regimen in patients with HIV infection, Curr HIV Res., Vol. 10, pp. 661-72 (2012) (“Calza”); Thompson M A et al., Antiretroviral Treatment of Adult HIV Infection: 2012 Recommendations of the International Antiviral Society—USA Panel, JAMA, Vol. 308, pp. 387-402 (2012) (“Thompson”). Factors influencing the likelihood of success include the duration of successful suppression prior to the regimen simplification and the extent to which patients are adherent to their simplified regimens. Calza. Although it has also been suggested that some patients may fail because of variability in trough concentrations of protease inhibitors, this has not been substantiated in rigorously conducted studies. Boffito M et al., Intra-individual variability in lopinavir plasma trough concentrations supports therapeutic drug monitoring, AIDS, Vol. 17, pp. 1107-1108 (2013). Other concerns that have been raised include the ability of HIV-1 protease inhibitors to achieve suppressive levels in the central nervous system. Thompson. The current consensus appears to be that this approach should be reserved for specific patient populations in which considerations related to chronic nucleoside toxicity and/or adherence to complex antiretroviral regimens are dominant. In these situations, the importance of adherence and of close monitoring of plasma HIV-1 RNA levels has been emphasized. In the case of HIV-1 protease inhibitor maintenance therapy, reestablishment of control of retroviral replication has generally been achieved by resumption of combination therapy.

Also, there is an interest in the development of infrequently administered therapy both as a treatment and a prevention strategy. The Long-Acting Antiretroviral Treatment Enabling (LATTE) study tested a combination of two oral antiretroviral drugs, the non-nucleoside reverse transcriptase inhibitor rilpivirine and the new integrase inhibitor GSK1265744. Spreen W R et al., Long-acting injectable antiretrovirals for HIV treatment and prevention, CURB OPIN HIV AIDS, Vol. 8(6):565571 (2013). Furthermore, a long-acting injectable medication can be an effective approach to circumvent the need for daily medication adherence and/or chronic nucleoside toxicity.

Further improvement in care may be realized with the development of new antiretroviral agents and methods of use that better or more completely suppress viral load, exhibit minimal drug or food interactions, reduce chronic toxicities associated with existing therapies, and permit dosing to be infrequent and flexible.

Early efforts to use monoclonal antibodies to suppress HIV replication were largely unsuccessful, and despite nearly two decades of human studies, monoclonal antibodies have not found a significant role in HIV prevention or therapy. However, recent studies with broadly neutralizing antibodies targeting the V3 region of HIV gp120 envelope and the approval of ibalizumab for salvage patients have renewed interest into monoclonal antibodies as therapeutic agents.

The use of monoclonal antibodies targeting the HIV entry co-receptor, CCR5, provides a novel class of potential therapeutic agents. PRO 140 acts by binding CCR5 on hematopoietic cells and preventing viral entry whereas the current antiretroviral agents target viral replication targets in the HIV life cycle. Previous small molecule inhibitors of CCR5, vicriviroc and maraviroc, have shown inferior efficacy in phase 3 trials in both naive and salvage patients compared to agents that interfere with the viral life cycle.

These agents are allosteric inhibitors of HIV fusion with cell membranes and have agonist activity resulting in activation of downstream tyrosine kinases triggering off target side effects. In contrast, PRO 140 is a competitive inhibitor antagonist of HIV recognition of CCR5 with no agonist activation of tyrosine kinases. Current antiretroviral agents are used in combination regimens due to the rapid development of resistance associated with monotherapy with these agents. PRO 140, a CCR5 co-receptor antagonist, presents a high genetic barrier to resistance and its unique mechanism of action to block HIV-1 entry supports its use as monotherapy for HIV-1 infection. Additionally, PRO 140 offers several potential advantages over existing therapies in terms of infrequent weekly dosing, favorable tolerability, and limited drug-drug or -food interactions.

CCR5 co-receptor antagonists represent an emerging antiretroviral treatment class and the first to target a host molecule. CCR5 is a chemokine receptor that mediates activation and migration of T cells and other leukocytes. CCR5 also binds the HIV-1 envelope glycoprotein gp120 and serves as a portal for HIV-1 entry into CD4⁺ cells. Lederman M M, Penn-Nicholson A, Cho M, Mosier D., Biology of CCR5 and its role in HIV infection and treatment. JAMA. 2006; 296 (7):815-26. CCR5-using (R5) viruses typically mediate transmission and then predominate through the progression to symptomatic disease. Viruses can use an alternative chemokine receptor, CXCR4, either exclusively or in addition to CCR5. CXCR4-using viruses may be present early on but tend to become apparent in an increasing percentage of subjects in later phases of disease. Dean M, Carrington M, Winkler C, et al., Genetic restriction of HIV-1 infection and progression to AIDS by a deletion allele of the CKR5 structural gene, Hemophilia Growth and Development Study, Multicenter AIDS Cohort Study, Multicenter Hemophilia Cohort Study, San Francisco City Cohort, ALIVE Study. SCIENCE 1996;273 (5283):1856-62; Moyle G J, Wildfire A, Mandalia S, et al., Epidemiology and predictive factors for chemokine receptor use in HIV-1 infection. J. INFECT DIS. 2005; 191(6):866-72; Wilkin T J, Su Z, Kuritzkes D R, et al., HIV type 1 chemokine coreceptor use among antiretroviral-experienced patients screened for a clinical trial of a CCR5 inhibitor: AIDS Clinical Trial Group A5211, CLIN. INFECT. DIS. 2007;44 (4):591-5; and Brumme Z L, Goodrich J, Mayer H B, et al., Molecular and clinical epidemiology of CXCR4-using HIV-1 in a large population of antiretroviral-naive individuals, J. INFECT. DIS. 2005;192 (3):466-74).

PRO 140 is a humanized CCR5 monoclonal antibody (mAb) that potently inhibits R5 viruses and synergizes with small-molecule CCR5 antagonists in laboratory studies. Murga J, Franti M, Pevear D C, Maddon P J, Olson W C, Potent antiviral synergy between monoclonal antibody and small-molecule CCR5 inhibitors of human immunodeficiency virus type 1, ANTIMICROBIAL AGENTS AND CHEMOTHERAPY, 2006; 50(10):3289-96.; Trkola A, Ketas T J, Nagashima K A, et al., Potent, broad-spectrum inhibition of human immunodeficiency virus type 1 by the CCR5 monoclonal antibody PRO 140, J. VIROL. 2001; 75 (2):579-88). PRO 140 does not inhibit CXCR4-using viruses.

PRO 140 binds to the N terminus (Nt) and the extracellular loop 2 (ECL2) domain of the CCR5 cell surface receptor that HIV-1 uses to gain entry to a cell. PRO 140 binding to CCR5 blocks the final phase of viral binding to the cell surface prior to fusion of the viral and cell membranes. PRO 140 has been administered intravenously or subcutaneously to 174 HIV-1 infected individuals in Phase VII studies of safety, tolerability, pharmacokinetics and pharmacodynamics. Jacobson J M et al., Study of the CCR5 monoclonal antibody PRO 140 administered intravenously to HIV-infected adults, Antimicrob Agents Chemother., Vol. 54, pp. 4137-42 (2010) (“Jacobson 2010”). The drug has been well tolerated following administration of single doses of 0.5 to 5 mg/kg or up to three weekly doses of up to 324 mg. Single subcutaneous doses of 324 mg have resulted in drops in plasma HIV-1 RNA levels of approximately 1.0 log₁₀. Repetitive weekly administration of this dose of PRO 140 has been associated with drops in plasma HIV-1 RNA levels of approximately 1.5 log₁₀. Serum concentrations of PRO 140 above the IC₅₀ for clinical isolates of HIV-1 are maintained for at least 2 weeks following a single dose of 324 mg. Plasma HIV-1 RNA levels rise to baseline levels as PRO 140 is cleared from the plasma and, presumably, other compartments.

Previously, an intravenous (IV) form of PRO 140 was tested as monotherapy in HIV-1 subjects with only R5 virus detectable. Jacobson J M, Saag M S, Thompson M A, et al., Antiviral activity of single-dose PRO 140, a CCR5 monoclonal antibody, in HIV-infected adults, J. INFECT. DIS. 2008; 198:1345-52 (“Jacobson 2008”). Single doses, ranging up to 5 mg/kg, were generally well tolerated relative to placebo and demonstrated potent and prolonged antiviral activity, with a 1.83 log₁₀ mean reduction in HIV-1 RNA observed at 5 mg/kg. These findings supported development of subcutaneous (SC) formulations with the potential for patient self-administration. Additional studies investigating PRO 140 SC formulations for use as monotherapy in HIV-1 subjects with only R5 virus detectable are underway.

Additional therapies, beyond HAART, for the ongoing treatment of HIV-1 infected subjects that are effective in reducing plasma viremia to levels that are undetectable in conventional, or standard, assays (i.e., <50 copies/mL) are needed. Additional therapies that may improve HIV-1 infected subjects' quality of life by reducing undesirable side effects associated with currently available therapies and ease therapy regimen adherence are also needed. Therapies that might address such current needs, and do so in a monotherapy format, are also highly desirable for reasons of ease, simplicity, and cost.

Further, as noted above HAART does not eradicate HIV-1 and long-term morbidities and CNS (HAND) problems still occur. Accordingly, additional approaches and therapies that might further reduce or maintain HIV-1 viral loads in a subject at actual undetectable viral loads (0 viral copies per mL of plasma as measured by SCA), extremely low viral loads (i.e., less than or equal to 1 viral copy per mL of plasma as measured by SCA), very low viral loads (i.e., less than or equal to 5 viral copies per mL of plasma as measured by SCA), or low viral loads (i.e., less than or equal to 10 viral copies per mL of plasma as measured by SCA), or be even more effective at reducing or maintaining HIV-1 viral loads at or below conventionally undetectable levels (i.e., <50 copies/mL), or reduce or maintain HIV-1 viral loads in a subject at actual undetectable viral loads, extremely low viral loads, very low viral loads, or low viral loads for prolonged periods of time (e.g., four (4) weeks or more, five (5) weeks or more, six (6) weeks or more, seven (7) weeks or more, eight (8) weeks or more, nine (9) weeks or more, ten (10) weeks or more, eleven (11) weeks or more, twelve (12) weeks or more, thirteen (13) weeks or more, fourteen (14) weeks or more, fifteen (15) weeks or more, sixteen (16) weeks or more, seventeen (17) weeks or more, eighteen (18) weeks or more, nineteen (19) weeks or more, twenty (20) weeks or more, twenty-one (21) weeks or more, twenty-two (22) weeks or more, twenty-three (23) weeks or more, twenty-four (24) weeks or more, twenty-five (25) weeks or more, twenty-six (26) weeks or more, or for periods of one (1), two (2), three (3), four (4), five (5), six (6), seven (7), eight (8), nine (9), ten (10), eleven (11), or twelve (12) months or more, or one, two, three, four, or five years or more) are needed.

Improved therapy modalities, including monotherapy, that may also provide a functional cure to HIV-1 infected patients, and that include suppressing viral load levels to actual undetectable viral loads (0 viral copies per mL of plasma as measured by SCA), extremely low viral loads (i.e., less than or equal to 1 viral copy per mL of plasma as measured by SCA), very low viral loads (i.e., less than or equal to 5 viral copies per mL of plasma as measured by SCA), or low viral loads (i.e., less than or equal to 10 viral copies per mL of plasma as measured by SCA), or be even more effective at reducing or maintaining HIV-1 viral loads at or below conventionally undetectable levels (i.e., <50 copies/mL) are needed. Such therapies not only represent major progress in the effective treatment of HIV-1 and improved quality of life for HIV-1 infected subjects, but may also reduce problems associated with long term toxicities related to HAART and may also represent public health advances because such therapies translate into improved prevention of HIV-1 transmission to uninfected subjects.

### BRIEF SUMMARY

The present invention arises out of the unexpected and surprising discovery that certain R5 virus tropic HIV-1 subjects with viral load effectively controlled using HAART, i.e., subjects having less than 50 viral copies/mL (<50 cp/mL), may be substantially more susceptible than others to effective monotherapy treatment using anti-CCR5 agents, such as PRO 140 mAbs. These certain R5 virus tropic HIV-1 subjects may be, in part, identified before, prepared by administration of higher doses of anti-CCR5 agents, and/or assessed during treatment using an SCA. Further, certain R5 virus tropic HIV-1 subjects may be given one or more high doses of an anti-CCR5 agent, such as PRO 140, in order to maximally suppress existing low level viremia before, at the beginning of, or during monotherapy treatment.

The present inventor determined that the level of viral suppression, including viral suppression below the conventional standard of less than 50 viral copies/mL (<50 cp/mL), prior to, upon initiation of, or during monotherapy treatment using anti-CCR5 agents, such as PRO 140 mAbs, may be used to effectively prognosticate whether certain R5 virus tropic HIV-1 subjects may be more or less responsive to monotherapy treatment. Generally, it was found that the more virally suppressed the R5 virus tropic HIV-1 subject is below the conventional standard of less than 50 viral copies/mL, the better the likelihood of their success on monotherapy treatment using anti-CCR5 agents, such as PRO 140 mAbs. For this reason, the present inventor developed a new approach to best ensure the subjects' success using monotherapy success by driving HIV-1 viral loads in R5 virus tropic HIV-1 subjects to maximal suppression.

The inventor determined that increasing the dose of anti-CCR5 agents, such as PRO 140 mAbs, is an effective approach to achieve further suppression of viral loads even in those HIV-1 infected subjects conventionally understood to be fully virally suppressed well below the conventional standard of less than 50 viral copies/mL (<50 cp/mL). And this approach may be used at any or all of prior to, upon initiation of, and during monotherapy to promote maximal viral load suppression. For example, administration of PRO 140 mAbs in higher doses, such as in amounts of 525 mg or 700 mg, can be used to suppress viral loads in HIV-1 infected patients to actual undetectable, extremely low, very low, or low levels, or to other levels between low levels and conventionally undetectable levels of <50 cp/mL. The inventor has determined that administration of higher doses increases the number HIV-1 subjects who are likely to respond to, and benefit from, monotherapy treatment using anti-CCR5 agents, such as PRO 140 mAbs. Additionally, the inventor has determined that administration of higher doses reduces, or shortens, the amount of time required to determine if a certain R5 virus tropic HIV-1 subject with viral load effectively controlled using HAART, i.e., subjects having less than 50 viral copies/mL (<50 cp/mL), is likely to respond to, and benefit from, monotherapy treatment using anti-CCR5 agents, such as PRO 140 mAbs.

Here, the present inventor found that certain R5 virus tropic HIV-1 subjects initiating anti-CCR5 agent monotherapy at a dose of 350 mg and having, for example, actual undetectable viral loads (0 viral copies per mL of plasma as measured by SCA) or extremely low viral loads (i.e., less than or equal to 1 viral copy per mL of plasma as measured by SCA) are far more likely, and up to about four (4) times more likely, to experience prolonged or unlimited periods of time with actual undetectable viral loads, extremely low viral loads, or conventionally undetectable viral loads (i.e., <50 viral copies per mL of plasma) than are R5 virus tropic HIV-1 subjects that initiate anti-CCR5 agent monotherapy having greater viral loads. Thus, the present invention may provide, for the first time, mechanisms and approaches to provide certain R5 virus tropic HIV-1 subjects with a monotherapy functional cure for HIV-1 infected subjects.

The present invention rebuts the conventional understanding of successful viral suppression as being assessed as conventionally undetectable viral loads (i.e., <50 viral copies per mL of plasma). The present invention re-visits and overturns conventional understanding to newly separate and distinguish between conventionally “viral suppressed” subjects to identify and treat those certain R5 virus tropic HIV-1 subjects not only most likely to succeed on an anti-CCR5 agent monotherapy, such as PRO 140, therapeutic regimen, but to experience prolonged viral suppression and, potentially, a monotherapy functional cure. The desire and long-standing need for such a therapeutic option is evidenced by, among other things, the fact that R5 virus tropic HIV-1 subjects who are successfully treated using HAART according to conventional standards (i.e., who have a viral load <50 copies/mL) have been, nonetheless, willing to stop the HAART treatment regime in order to find out if they might further benefit from an anti-CCR5 agent monotherapy, such as PRO 140, therapeutic regimen.

Implementing their new found and unconventional approaches, the present inventors have developed novel methods involving SCAs or high dosages, or the combination of using SCAs and high dosages, to provide methods and kits capable of assessing and prognosticating HIV-1 subject susceptibility to monotherapy treatment and new methods to treat such subjects for maximal success using an anti-CCR5 agent such as, for example, PRO 140. Further, the present inventors have developed novel methods involving SCAs or high dosages, or the combination of using SCAs and high dosages that may allow these R5 virus tropic HIV-1 subjects who are successfully treated using HAART according to conventional standards to safely withdraw from HAART and, further, to enjoy the avoidance of toxicities and long-term side effects associated with HAART and the improved quality of life associated with an anti-CCR5 agent monotherapy, such as PRO 140, therapeutic regimen.

### BRIEF DESCRIPTION OF THE DRAWINGS

FIG. 1A, FIG. 1B, and FIG. 1C show four (4) week data sets for fifty-four (54) R5 virus tropic HIV-1 subjects with viral load effectively controlled using HAART following their switch to subcutaneous (SC) PRO 140 monotherapy treatment.

FIG. 2 shows the Emax analysis of antiviral data generated with intravenous (IV) and subcutaneous (SC) PRO 140.

FIG. 3 shows the time to loss of virologic response for 16 subjects over 900 days for a CD01 extension study.

FIG. 4A and FIG. 4B show interim results achieved on the CD03 low (350 mg) dose versus higher (525 mg) dose monotherapy study.

### DETAILED DESCRIPTION

The present invention arises out of the unexpected and surprising discovery that certain R5 virus tropic HIV-1 subjects with viral load effectively controlled using HAART, i.e., subjects having less than 50 viral copies/mL (<50 cp/mL), may be substantially more susceptible than others to effective monotherapy treatment using anti-CCR5 agents, such as PRO 140 mAbs. The present invention also relates to methods of identifying, preparing, and/or treating certain R5 virus tropic HIV-1 subjects who are most likely to be responsive to an anti-CCR5 agent monotherapy, such as PRO 140, therapeutic regimen by using either or both of an SCA and high dosages such as, for example, 700 mg.

The following exemplary screening methods relate to identification and treatment of patent subpopulations in studies designed to evaluate the efficacy, safety, tolerability, and success of PRO 140 monotherapy for the maintenance of viral suppression in R5 virus tropic HIV-1 subjects who are stable on combination antiretroviral therapy. The first screening method involves, among other criteria, the use of an SCA to help determine a subject's eligibility for study participation and to prognosticate success. Application of the first screening method and corresponding study results are provided, for example, in Example 1. The second screening method contemplates administration of a high-dose of PRO 140, (e.g., greater than about 350 mg, 437 mg, 525 mg, 700 mg, 787 mg, etc.) and does not necessarily involve the use of an SCA to help determine a subject's eligibility for study participation and to prognosticate success. It is contemplated that administration of a high-dose of PRO 140 will maximize viral suppression in R5 virus tropic HIV-1 subjects prior to, at the onset of, and/or during treatment, and that such maximal suppression including, for example, to levels ≤1 copy per mL will increase the subject's likelihood of therapeutic success.

Screening

In a preferred embodiment, one inclusion criterion for the present study requires each patient to have a conventionally undetectable viral load for the 12 months prior to enrollment (e.g., <50 cp/mL). As only HIV patients who have R5 virus exclusively can benefit from PRO 140, each patient is required to take a DNA TROFILE® test prior to enrollment in the study.

In a preferred embodiment, the DNA TROFILE® test is used, but other tropism assays may also be used.

In addition to measuring for viral tropism, subjects may take a single copy assay (SCA) test to determine viral load counts. The SCA is more sensitive to determination of viral load counts equal to or less than 50 copies/mL (plasma), and may be used to determine a viral load count in any integer value ≤50. For example, the SCA may be used to determine viral load counts equal to or less than 50 copies/mL, equal to or less than 45 copies/mL, equal to or less than 40 copies/mL, equal to or less than 35 copies/mL, equal to or less than 30 copies/mL, equal to or less than 25 copies/mL, equal to or less than 20 copies/mL, equal to or less than 15 copies/mL, equal to or less than 10 copies/mL, equal to or less than 9 copies/mL, equal to or less than 8 copies/mL, less than 7 copies/mL, equal to or less than 6 copies/mL, equal to or less than 5 copies/mL, equal to or less than 4 copies/mL, equal to or less than 3 copies/mL, less than 2 copies/mL, equal to or less than 1 copy/mL, and equal to or greater than 0 copies/mL but equal to or less than less than 1 copy/mL.

In a preferred embodiment, the SCA is the bioMONTR® Labs HIV-1 SuperLow Assay (Single-copy HIV-1 RNA Assay), but other assays may also be used. The bioMONTR® Labs HIV-1 SuperLow Assay was developed using a modified protocol of a CE marked commercial kit.

The bioMONTR® Labs HIV-1 SuperLow Assay method is described as follows. See McClernon, A. M. et al, New HIV-1 SuperLow Assay for Viral Load Monitoring, bioMONTR Labs website, available at: http://www.biomontr.com/. First, Viral subtype B RNA in HIV-1 negative human plasma and panel members from the 2011 Human Immunodeficiency Virus RNA EQA Programme (available from Quality Control for Molecular Diagnostics (QCMD)) were extracted on bioMerieux's (Durham, N.C.) NucliSens® easyMAG® platform. Second, extracts were analyzed using bioMONTR's proprietary HIV-1 SuperLow Assay described here-in which utilizes components of bioMerieux's commercially available (RUO) EasyQ® HIV-1 v2.0. Third, testing on the HIV-1 SuperLow Assay was performed using a 2.0 mL sample input, with the exception of QCMD panel samples which were 1.0 mL each. The acceptable maximum allowable standard deviation (SD) criteria of ±0.50 log₁₀ c/mL was established based on criteria by the HHS Panel on Antiretroviral Guidelines. Third, for determination of Precision and the Limit of Detection (LOD), dilutions of Virology Quality Assurance (VQA) viral standards were made in HIV-1 negative human plasma yielding dilutions of approximately 3, 6, 12, 24, 48, 72, and 96 c/mL. At least 27 replicates of each concentration were tested using a single lot of extraction and amplification reagents. Probit analysis to determine the 95% hit rate using Percent Detected (PD) values at each dilution. Excel 2007 (Microsoft) function NORMSINV (z) was used to translate PD values to probit values. Fourth, for testing analytical measurement range, Virology Quality Assurance (VQA) stock material at 107 log was diluted 1:10 serially 5× in normal HIV-1 negative human plasma to yield dilutions of 1:10, 1:100, 1:1,000, 1:10,000 and 1:100,000 and tested in a single run.

It is reported that the bioMONTR® Labs HIV-1 SuperLow Assay demonstrated impressive hit rates: 95% at 15 c/mL and 70% at 7 c/mL. The HIV-1 SuperLow Assay has a reportable range of 2 to 10,000,000 c/mL. The assay was verified to have acceptable precision and accuracy well within the range considered to be statistically significant for clinical interpretation. As expected, the precision decreases when the concentration of the analyte decreases. All results obtained from QCMD Panel Members were as expected and quantitative performance on paired samples were within 0.5 log units of the median. bioMONTR's Quantitative Consensus Panel Score ranked in the 73rd percentile of all datasets (i.e., 27% of all datasets had the same, or better, score). The assay produced reportable quantitative results as low as 3 c/mL for samples previously reported as <50 c/mL.

Alternative considerations for eligibility screening may include the contemplated dosage of PRO 140 to be delivered. Increasing doses of PRO 140 are increasingly effective at reducing viral load in treated subjects. Accordingly, it is contemplated that sufficiently high doses of PRO 140 prior to, upon initiation of, or during study treatment will rapidly or ultimately result in actual undetectable viral loads (as measured by SCA), extremely low viral loads (i.e., less than or equal to 1 viral copy per mL of plasma as measured by SCA), very low viral loads (i.e., less than or equal to 5 viral copies per mL of plasma as measured by SCA), or low viral loads (i.e., less than or equal to 10 viral copies per mL of plasma as measured by SCA), or be even more effective at reducing or maintaining HIV-1 viral loads at or below conventionally undetectable levels (i.e., <50 copies/mL). For example, in one preferred embodiment, a dosage amount of 525 mg of PRO 140 is provided in two 1.5 mL subcutaneous injections, wherein each mL of formulation has a PRO 140 concentration of about 175 mg/mL. For example, in another preferred embodiment, a dosage amount of 700 mg of PRO 140 is provided in two 2.0 mL subcutaneous injections, wherein each mL of formulation has a PRO 140 concentration of about 175 mg/mL. In still other embodiments, a dosage amount of one of 350 mg, 437 mg, 525 mg, 700 mg, 787 mg, etc., may be delivered in one or more injections and include a formulation having a concentration of greater or less than 175 mg/mL. For example, formulations of PRO 140 may have a concentration in an amount greater than about or equal to 100 mg/mL and less than about or equal to 200 mg/mL or in an amount of greater than about or equal to 162 mg/mL to about 175 mg/mL, or in an amount of greater than about or equal to 175 mg/mL to about 180 mg/mL. The PRO 140 concentration may have a concentration of about or equal to 185 mg/mL, about or equal to 180 mg/mL, about or equal to 175 mg/mL, about or equal to 170 mg/mL, or about or equal to 165 mg/mL. More specifically, the protein is present in the formulations in an amount of 150 mg/mL to 200 mg/mL, or in an amount of 1 mg/mL increments from 150 mg/mL up to 200 mg/mL, e.g., 151 mg/mL, 152 mg/mL, 152 mg/mL, 153 mg/mL, 154 mg/mL, 155 mg/mL, 156 mg/mL, 157 mg/mL, 158 mg/mL, 159 mg/mL, 160 mg/mL, 161 mg/mL, 162 mg/mL, 163 mg/mL, 164 mg/mL, 165 mg/mL, etc. While PRO 140 is specifically identified here, other proteins, including but not limited to other anti-CCR5 agents, are also contemplated for use with the present invention.

In one embodiment, sufficiently high doses of PRO 140 may be administered together with a subject's current ongoing therapy for a period of time in order to decrease the subject's viral load count to actual undetectable viral loads (as measured by SCA), extremely low viral loads (i.e., less than or equal to 1 viral copy per mL of plasma as measured by SCA), very low viral loads (i.e., less than or equal to 5 viral copies per mL of plasma as measured by SCA), or low viral loads (i.e., less than or equal to 10 viral copies per mL of plasma as measured by SCA), or at any other specified or target viral load level below 50 viral copies per mL of plasma as measured by SCA before initiation of monotherapy. Preferably, administration of these sufficiently high doses of PRO 140 will decrease the subject's viral load count to actual undetectable viral loads (as measured by SCA), extremely low viral loads (i.e., less than or equal to 1 viral copy per mL of plasma as measured by SCA), or at any other specified or target viral load level below 50 viral copies per mL of plasma as measured by SCA before initiation of monotherapy.

It is contemplated that increased doses of PRO 140 may also be used during the course of monotherapy treatment on either a temporary or ongoing basis to further suppress a subject's viral load count if and as needed to maintain a target viral load level. For example a higher dose of PRO 140 may be used one or more times to reduce a viral load count that is elevated above a specified or target viral load level. Alternatively, a higher dose of PRO 140 may be used one or more times to maintain a viral load count that is elevated above a specified or target viral load level. It is also contemplated that the amount of PRO 140 dosed may fluctuate during monotherapy on a subject-by-subject basis to achieve suitable viral load suppression in a particular subject by administering no more PRO 140 than necessary. That is, a subject's responsiveness to the PRO 140 monotherapy, as measured for example by viral load, may be used to determine an appropriate or suitable dosing schedule for that subject.

In a preferred embodiment, anti-CCR5 formulations, such as PRO 140 formulations, are delivered as concentrated protein formulations with concentrations of, for example, 162 mg/mL, 170 mg/mL, 175 mg/mL, 180 mg/mL, 185 mg/mL, 190 mg/mL, 195 mg/mL, 200 mg/mL, etc. The formulations may be administered intravenously or subcutaneously. The formulations may be administered as one or more contemporaneous split doses to deliver the total dosage payload. For example, the formulations may be administered as one or more contemporaneous split doses such as 2 injections each containing 2mLs of PRO 140 formulation concentrated to 175 mg/mL to deliver the total dosage payload of 700 mg. The doses may be administered at one or more of prior to treatment, upon initiation of treatment, and during treatment.

In one embodiment, it is contemplated that a set dosage amount, once established for a particular treatment regime, will not change over the course of treatment. In another embodiment, it is contemplated that a dosage amount may vary based on a subject's expected or known viral load count. In still another embodiment, it is contemplated that a subject may be administered varied dosage amounts over the course of treatment. In another embodiment, it is contemplated that a subject may receive a higher dose before or upon initiation of treatment than is administered during treatment. In another embodiment, it is contemplated that a higher dose may be administered during treatment in response to an increase in viral load count.

### Methods of Use

In one aspect, the present disclosure provides methods of screening HIV-1-infected subjects and treating or preventing HIV-1 infection comprising administering to a subject having actual undetectable viral loads (0 viral copies per mL of plasma as measured by SCA), extremely low viral loads (i.e., less than or equal to 1 viral copy per mL of plasma as measured by SCA), very low viral loads (i.e., less than or equal to 5 viral copies per mL of plasma as measured by SCA), or low viral loads (i.e., less than or equal to 10 viral copies per mL of plasma as measured by SCA), or at any other specified or target viral load level below 50 viral copies per mL of plasma as measured by SCA, in need thereof a competitive inhibitor to a CCR5 cell receptor.

In one embodiment, the present invention relates to a method of screening comprising, determining the presence of non-CCR5 viral tropism in an HIV-1-infected subject. In another embodiment, the method of screening comprises, using a SCA to determine the viral load, or level of viremia, of an HIV-1-infected subject. In a preferred embodiment, the method of screening comprises determining the presence of non-CCR5 viral tropism in an HIV-1-infected subject and using a SCA to determine the viral load, or level of viremia, of an HIV-1-infected subject.

In another embodiment, the present invention provides a method for effectively maintaining low, very low, extremely low, or actually undetectable HIV-1 viral load, or an HIV-1 viral load at any other specified or target viral load level below 50 viral copies per mL of plasma, as measured by SCA in an HIV-1-infected subject using monotherapy. In another embodiment, the present invention provides a method for effectively maintaining low, very low, extremely low, or actually undetectable HIV-1 viral load, or an HIV-1 viral load at any other specified or target viral load level below 50 viral copies per mL of plasma, in an HIV-1-infected subject using monotherapy and facilitating treatment by use of one or more SCAs before, upon initiation, and during treatment. In another embodiment, the present invention provides a method for effectively maintaining low, very low, extremely low, or actually undetectable HIV-1 viral load, or an HIV-1 viral load at any other specified or target viral load level below 50 viral copies per mL of plasma, in an HIV-1-infected subject using monotherapy and facilitating treatment by use of one or more high doses of an anti-CCR5 agent before, upon initiation, and during treatment. In a preferred embodiment, the present invention provides a method for effectively maintaining extremely low or actually undetectable HIV-1 viral load, or an HIV-1 viral load at any other specified or target viral load level below 50 viral copies per mL of plasma, in an HIV-1-infected subject using monotherapy and facilitating treatment by use of one or more high doses of PRO 140 before, upon initiation, and during treatment. In still another embodiment, the present invention provides a method for effectively maintaining low, very low, extremely low, or actually undetectable HIV-1 viral load, or an HIV-1 viral load at any other specified or target viral load level below 50 viral copies per mL of plasma, in an HIV-1-infected subject using monotherapy and facilitating treatment by use of one or more SCAs before, upon initiation, and during treatment and one or more high doses of an anti-CCR5 agent, such as PRO 140, before, upon initiation, and during treatment.

In one embodiment, the present disclosure provides a method of preventing HIV-1 progression or infection comprising administering to a subject in need thereof a competitive inhibitor to a CCR5 cell receptor, wherein the competitive inhibitor binds to the ECL-2 loop of the CCR5 cell receptor. In a further embodiment, the competitive inhibitor competes with CCL5 for binding to the CCR5 cell receptor. In a further embodiment, the competitive inhibitor competes for binding with the monoclonal antibody PRO 140 or a binding fragment thereof.

In one embodiment, the present disclosure provides a method of preventing HIV-1 progression or infection comprising administering to a subject in need thereof: (a) a PRO 140 antibody, or binding fragment thereof; (b) a nucleic acid encoding a PRO 140 antibody, or binding fragment thereof; (c) a vector comprising a nucleic acid encoding a PRO 140 antibody, or binding fragment thereof; or (d) a host cell comprising (i) a PRO 140 antibody, or binding fragment thereof, (ii) a nucleic acid encoding a PRO 140 antibody, or binding fragment thereof, or (iii) a vector comprising a nucleic acid encoding a PRO 140 antibody, or binding fragment thereof. In the aforementioned embodiment, the PRO 140 antibody, or binding fragment thereof, may comprise, for example, a PRO 140 monoclonal antibody or a scFv.

In one embodiment, the present disclosure provides a method of preventing HIV-1 progression or infection comprising administering to a subject in need thereof a PRO 140 antibody, or binding fragment thereof

In any of the aforementioned embodiments, preventing HIV-1 progression, or treating an HIV-1 infected subject may comprise maintaining the HIV-1 viral load below conventional undetectable levels (i.e., <50 copies/mL), or at low, very low, extremely low, or actually undetectable levels. For example, the HIV-1 viral load levels may be less than or equal to 0 copies/mL, less than or equal to 1 copy/mL, less than or equal to 2 copies/mL, less than or equal to 3 copies/mL, less than or equal to 4 copies/mL, less than or equal to 5 copies/mL, less than or equal to 6 copies/mL, less than or equal to 7 copies/mL, less than or equal to 8 copies/mL, less than or equal to 9 copies/mL, less than or equal to 10 copies/mL, less than or equal to 11 copies/mL, less than or equal to 12 copies/mL, less than or equal to 13 copies/mL, less than or equal to 14 copies/mL, less than or equal to 15 copies/mL, less than or equal to 16 copies/mL, less than or equal to 17 copies/mL, less than or equal to 18 copies/mL, less than or equal to 19 copies/mL, less than or equal to 20 copies/mL, less than or equal to 21 copies/mL, less than or equal to 22 copies/mL, less than or equal to 23 copies/mL, less than or equal to 24 copies/mL, less than or equal to 25 copies/mL, less than or equal to 26 copies/mL, less than or equal to 27 copies/mL, less than or equal to 28 copies/mL, less than or equal to 29 copies/mL, less than or equal to 30 copies/mL, less than or equal to 31 copies/mL, less than or equal to 32 copies/mL, less than or equal to 33 copies/mL, less than or equal to 34 copies/mL, less than or equal to 35 copies/mL, less than or equal to 36 copies/mL, less than or equal to 37 copies/mL, less than or equal to 38 copies/mL, less than or equal to 39 copies/mL, less than or equal to 40 copies/mL, less than or equal to 41 copies/mL, less than or equal to 42 copies/mL, less than or equal to 43 copies/mL, less than or equal to 44 copies/mL, less than or equal to 45 copies/mL, less than or equal to 46 copies/mL, less than or equal to 47 copies/mL, less than or equal to 48 copies/mL, less than or equal to 49 copies/mL, or less than or equal to 50 copies/mL.

Also, in any of the aforementioned embodiments, preventing HIV-1 progression or maintaining viral suppression to below conventional undetectable levels (i.e., <50 copies/mL), or at low, very low, extremely low, or actually undetectable levels may comprise elevating or maintaining elevated CD4+cell counts in an HIV-1 infected subject. For example, such prevention may result in a treated subject having CD4 cell count greater than 600 cells/mm³, greater than 550 cells/mm³, greater than 500 cells/mm³, greater than 450 cells/mm³, greater than 400 cells/mm³, or greater than 350 cells/mm³.

### CCR5 Antagonists

In one aspect, the present disclosure relates to the use of CCR5 antagonists, i.e., anti-CCR5 agents, that target CCR5 receptor and act as competitive inhibitors to the CCR5 cell receptor without providing CCL5 agonist activity.

In one embodiment, the present disclosure provides for the use of a PRO 140 antibody, or binding fragment thereof, in treating or preventing HIV-1 infection. PRO 140 is a humanized monoclonal antibody described in U.S. Pat. Nos. 7,122,185 and 8,821,877, the contents of which are incorporated herein by reference in their entirety. PRO 140 is a humanized version of the murine mAb, PA14, which was generated against CD4⁺ CCR5⁺ cells. Olson et al., Differential Inhibition of Human Immunodeficiency Virus Type 1 Fusion, gp 120 Binding and CC-Chemokine Activity of Monoclonal Antibodies to CCR5, J. VIROL., 73: 4145-4155. (1999). PRO 140 binds to CCR5 expressed on the surface of a cell, and potently inhibits HIV-1 entry and replication at concentrations that do not appear to affect CCR5 chemokine receptor activity in vitro and in the hu-PBL-SCID mouse model of HIV-1 infection. Olson et al., Differential Inhibition of Human Immunodeficiency Virus Type 1 Fusion, gp 120 Binding and CC-Chemokine Activity of Monoclonal Antibodies to CCR5, J. VIROL., 73: 4145-4155. (1999); Trkola et al., Potent, Broad-Spectrum Inhibition of Human Immunodeficiency Virus Type 1 by the CCR5 Monoclonal Antibody PRO 140, J. VIROL., 75: 579-588 (2001).

Nucleic acids encoding heavy and light chains of the humanized PRO 140 antibody have been deposited with the ATCC. Specifically, the plasmids designated pVK-HuPRO140, pVg4-HuPRO140 (mut B+D+I) and pVg4-HuPRO140 HG2, respectively, were deposited pursuant to, and in satisfaction of, the requirements of the Budapest Treaty with the ATCC, Manassas, Va., U.S.A. 20108, on Feb. 22, 2002, under ATCC Accession Nos. PTA 4097, PTA 4099, and PTA 4098, respectively.

In a one embodiment, the methods disclosed herein comprise administering a humanized antibody designated PRO 140 or an antibody that competes with PRO 140 for binding to the CCR5 receptor, wherein the PRO 140 comprises (i) two light chains, each light chain comprising the expression product of the plasmid designated pVK:HuPRO140-VK (ATCC Deposit Designation PTA-4097), and (ii) two heavy chains, each heavy chain comprising the expression product of either the plasmid designated pVg4:HuPRO140 HG2-VH (ATCC Deposit Designation PTA-4098) or the plasmid designated pVg4:HuPRO140 (mut B+D+I)-VH (ATCC Deposit Designation PTA-4099). In a further embodiment, the PRO 140 is a humanized or human antibody that binds to the same epitope as that to which antibody PRO 140 binds. In another embodiment, the monoclonal antibody is the humanized antibody designated PRO 140.

In one embodiment of the methods described herein, the antibody or binding fragment thereof comprises a light chain of the antibody. In another embodiment, the antibody or binding fragment thereof comprises a heavy chain of the antibody. In a further embodiment, the antibody or binding fragment thereof comprises an Fab portion of the antibody. In a still further embodiment, the antibody or binding fragment thereof comprises an F(ab′)₂ portion of the antibody. In an additional embodiment, the antibody or binding fragment thereof comprises an Fd portion of the antibody. In another embodiment, the antibody or binding fragment thereof comprises an Fv portion of the antibody. In a further embodiment, the antibody or binding fragment thereof comprises a variable domain of the antibody. In a still further embodiment, the antibody or binding fragment thereof comprises one or more CDR domains of the antibody. In yet another embodiment, the antibody or binding fragment thereof comprises six CDR domains of the antibody.

The present disclosure also provides antibody or antibody fragment-polymer conjugates having an effective size or molecular weight that confers an increase in serum half-life, an increase in mean residence time in circulation (MRT), and/or a decrease in serum clearance rate over underivatized antibody fragments. Antibody fragment-polymer conjugates can be made by derivatizing the desired antibody fragment with an inert polymer. It will be appreciated that any inert polymer which provides the conjugate with the desired apparent size or which has the selected actual molecular weight is suitable for use in constructing antibody fragment-polymer conjugates of the invention.

In one embodiment, the competitive inhibitor to a CCR5 cell receptor, such as PRO 140, is administered with a pharmaceutically acceptable carrier. Examples of concentrated protein formulations suitable for use with the present invention are disclosed in U.S. patent application Ser. No. 13/582,243, now U.S. Pat. No. 9,956,165, the contents of which are fully incorporated by herein by this reference. Pharmaceutically acceptable carriers are well known to those skilled in the art. Such pharmaceutically acceptable carriers may include but are not limited to aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, saline, and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases, and the like.

The dose of the composition of the invention will vary depending on the subject and upon the particular route of administration used. Dosages can range from 0.1 to 100,000 μg/kg. Based upon the composition, the dose can be delivered continuously, such as by continuous pump, or at periodic intervals, e.g., on one or more separate occasions. Desired time intervals of multiple doses of a particular composition can be determined without undue experimentation by one skilled in the art.

In one embodiment of the instant methods, the antibody or binding fragment thereof is administered to the subject a plurality of times and each administration delivers from 0.01 mg per kg body weight to 50 mg per kg body weight of the antibody or binding fragment thereof to the subject. In another embodiment, each administration delivers from 0.05 mg per kg body weight to 25 mg per kg body weight of the antibody or binding fragment thereof to the subject. In a further embodiment, each administration delivers from 0.1 mg per kg body weight to 10 mg per kg body weight of the antibody or binding fragment thereof to the subject. In a still further embodiment, each administration delivers from 0.5 mg per kg body weight to 5 mg per kg body weight of the antibody or binding fragment thereof to the subject. In another embodiment, each administration delivers from 1 mg per kg body weight to 3 mg per kg body weight of the antibody or binding fragment thereof to the subject. In a another embodiment, each administration delivers about 2 mg per kg body weight of the antibody or binding fragment thereof to the subject.

In one embodiment, the antibody or binding fragment thereof is administered a plurality of times, and a first administration is separated from the subsequent administration by an interval of less than one week. In another embodiment, the first administration is separated from the subsequent administration by an interval of at least one week. In a further embodiment, the first administration is separated from the subsequent administration by an interval of one week. In another embodiment, the first administration is separated from the subsequent administration by an interval of two to four weeks. In another embodiment, the first administration is separated from the subsequent administration by an interval of two weeks. In a further embodiment, the first administration is separated from the subsequent administration by an interval of four weeks. In yet another embodiment, the antibody or binding fragment thereof is administered a plurality of times, and a first administration is separated from the subsequent administration by an interval of at least one month. In another embodiment, the antibody or binding fragment thereof is administered on an as-needed basis to reduce a spike in viral load and/or between any of the above-noted regular dosage intervals.

In a further embodiment, the antibody or binding fragment thereof is administered to the subject via intravenous (IV) infusion. In another embodiment, the antibody or binding fragment thereof is administered to the subject via subcutaneous (SC) injection. In another embodiment, the antibody or binding fragment thereof is administered to the subject via intramuscular (IM) injection.

While the present invention contemplates monotherapy, in one embodiment, the competitive inhibitor to a CCR5 cell receptor, such as PRO 140, is administered in combination with one or more other therapeutic molecules or treatment, such a cellular therapy, e.g., an autologous or allogeneic immunotherapy; a small molecule; or an inhibitor of CCR5/CCL5 signaling, such as maraviroc, vicriviroc, aplaviroc, SCH-C, TAK-779, PA14 antibody, 2D7 antibody, RoAb13 antibody, RoAb14 antibody, or 45523 antibody. In one embodiment, the methods disclosed herein comprise administering PRO 140 in combination with, for example, maraviroc, vicriviroc, aplaviroc, SCH-C, TAK-779, PA14 antibody, 2D7 antibody, RoAb13 antibody, RoAb14 antibody, or 45523 antibody.

In one embodiment, the competitive inhibitor to a CCR5 cell receptor, such as PRO 140, is administered in combination with one or more small molecules, such as SCH-C (Strizki et al., PNAS, 98: 12718-12723 (2001)); SCH-D (SCH 417670; vicriviroc); UK-427,857 (maraviroc; 1-[(4,6-dimethyl-5-pyrimidinyl) carbonyl]-4-[4-[2-methoxy-1(R)-4-(trifluoromethyl)phenyl]ethyl-3(S)-methyl-1-piperazinyli-4-methylpiperidine); GW873140; TAK-652; TAK-779; AMD070; AD101; 1,3,4-trisubstituted pyrrolidines (Kim et al., BIOORG. MED. CHEM. LETT., 15: 2129-2134 (2005)); modified 4-piperidinyl-2-phenyl-1-(phenylsulfonylamino)-butanes (Shah et al., BIOORG. MED. CHEM. LETT., 15: 977-982 (2005)); Anibamine TFA, Ophiobolin C, or 19,20-epoxycytochalasin Q (Jayasuriya et al., J. NAT. PROD., 67: 1036-1038 (2004)); 5-(piperidin-1-yl)-3-phenyl-pentylsulfones (Shankaran et al., BIOORG. MED. CHEM. LETT., 14: 3589-3593 (2004)); 4-(heteroarylpiperdin-1-yl-methyl)-pyrrolidin-1-yl-acetic acid antagonists (Shankaran et al., BIOORG. MED. CHEM. LETT., 14: 3419-3424 (2004)); agents containing 4-(pyrazolyl)piperidine side chains (Shu et al., BIOORG. MED. CHEM. LETT., 14: 947-52 (2004); Shen et al., BIOORG. MED. CHEM. LETT., 14: 935-939 (2004); Shen et al., BIOORG. MED. CHEM. LETT., 14: 941-945 (2004)); 3-(pyrrolidin-l-yl)propionic acid analogues (Lynch et al., Org. Lett., 5: 2473-2475 (2003)); [2-(R)-[N-methyl-N-(1-(R)-3-(S)-((4-(3-benzyl-1-ethyl-(1H)-pyrazol-5-yl)piperidin-1-yl)methyl)-4-(S)-(3-fluorophenyl)cyclopent-1-yl)amino]-3-methylbutanoic acid (MRK-1)] (Kumar et al., J. PHARMACOL. EXP. THER., 304: 1161-1171 (2003)); 1,3,4 trisubstituted pyrrolidines bearing 4-aminoheterocycle substituted piperidine side chains (Willoughby et al., BIOORG. MED. CHEM. LETT., 13: 427-431 (2003); Lynch et al., BIOORG. MED. CHEM. LETT., 12: 3001-3004 (2003); Lynch et al., BIOORG. MED. CHEM. LETT., 13: 119-123 (2003); Hale et al., BIOORG. MED. CHEM. LETT., 12: 2997-3000 (2002)); bicyclic isoxazolidines (Lynch et al., BIOORG. MED. CHEM. LETT., 12: 677-679 (2002)); combinatorial synthesis of CCR5 antagonists (Willoughby et al., BIOORG. MED. CHEM. LETT., 11: 3137-41 (2001)); heterocycle-containing compounds (Kim et al., BIOORG. MED. CHEM. LETT., 11: 3103-3106 (2001)); antagonists containing hydantoins (Kim et al., BIOORG. MED. CHEM. LETT., 11: 3099-3102 (2001)); 1,3,4 trisubstituted pyrrolidines (Hale et al., BIOORG. MED. CHEM. LETT., 11: 2741-2745 (2001)); 1-[N-(methyl)-N-(phenylsulfonyl)amino]-2-(phenyl)-4-(4-(N-(alkyl)-N-(benzyloxycarbonyl)amino)piperidin-1-yl)butanes (Finke et al., BIOORG. MED. CHEM. LETT., 11: 2475-2479 (2001)); compounds from the plant Lippia alva (Hedge et al., BIOORG. MED. CHEM. LETT., 12: 5339-5342 (2004)); piperazine-based CCR5 antagonists (Tagat et al., J. MED. CHEM., 47: 2405-2408 (2004)); oximino-piperidino-piperidine-based CCR5 antagonists (Palani et al., BIOORG. MED. CHEM. LETT., 13: 709-712 (2003)); rotamers of SCH 351125 (Palani et al., BIOORG. MED. CHEM. LETT., 13: 705-708 (2003)); piperazine-based symmetrical heteroaryl carboxamides (McCombie et al., BIOORG. MED. CHEM. LETT., 13: 567-571 (2003)); oximino-piperidino-piperidine amides (Palani et al., J. MED. CHEM., 45: 3143-3160 (2002)); Sch-351125 and Sch-350634 (Este, CURR. OPIN. INVESTIG. DRUGS., 3: 379-383 (2002)); 1-[(2,4-dimethyl-3-pyridinyl)carbonyl]-4-methyl-4-[3(S)-methyl-4-[1(S)-[4-(trifluoromethyl)phenyl]ethyl]-1-piperazinyl]-piperidine N1-oxide (Sch-350634) (Tagat et al., J. MED. CHEM., 44: 3343-3346 (2001)); 4-[(Z)-(4-bromophenyl)-(ethoxyimino)methyl]-1′-[(2,4-dimethyl-3-pyridinyl)carbonyl]-4′-methyl-1,4′-bipiperidine N-oxide (SCH 351125) (Palani et al., J. MED. CHEM., 44: 3339-3342 (2001)); 2(S)-methyl piperazines (Tagat et al., BIOORG. MED. CHEM. LETT., 11: 2143-2146 (2001)); piperidine-4-carboxamide derivatives (Imamura et al., BIOORG. MED. CHEM., 13: 397-416, 2005); 1-benzazepine derivatives containing a sulfoxide moiety (Seto et al., BIOORG. MED. CHEM. LETT., 13: 363-386 (2005)); anilide derivatives containing a pyridine N-oxide moiety (Seto et al., CHEM. PHARM. BULL. (Tokyo), 52: 818-829 (2004)); 1-benzothiepine 1,1-dioxide and 1-benzazepine derivatives containing a tertiary amine moiety (Seto et al., CHEM. PHARM. BULL. (Tokyo), 52: 577-590 (2004)); N-[3-(4-benzylpiperidin-1-yl)propyl]-N,N′-diphenylureas (Imamura et al., BIOORG. MED. CHEM., 12: 2295-2306 (2004)); 5-oxopyrrolidine-3-carboxamide derivatives (Imamura et al., CHEM. PHARM. BULL. (Tokyo), 52: 63-73 (2004); anilide derivatives with a quaternary ammonium moiety (Shiraishi et al., J. MED. CHEM., 43: 2049-2063 (2000)); AK602/ONO4128/GW873140 (Nakata et al., J. VIROL., 79: 2087-2096 (2005)); spirodiketopiperazine derivatives (Maeda et al., J. BIOL. CHEM., 276: 35194-35200 (2001); Maeda et al., J. VIROL., 78: 8654-8662 (2004)); and selective CCR5 antagonists (Thoma et al., J. MED. CHEM., 47: 1939-1955 (2004)).

In one embodiment, the competitive inhibitor to a CCR5 cell receptor, such as PRO 140, is administered in combination with one or more of SCH-C, SCH-D (SCH 417670, or vicriviroc), UK-427,857 (maraviroc), GW873140, TAK-652, TAK-779 AMD070, or AD101. See U.S. Pat. No. 8,821,877.

In one embodiment, the competitive inhibitor to a CCR5 cell receptor, such as PRO 140, exhibits synergistic effects when administered in combination with one or more other therapeutic molecules or treatment, such as a cellular therapy, a small molecule, a chemotherapeutic, or an inhibitor of CCR5/CCL5 signaling. “Synergy” between two or more agents refers to the combined effect of the agents which is greater than their additive effects. Synergistic, additive, or antagonistic effects between agents may be quantified by analysis of the dose-response curves using the Combination Index (CI) method. A CI value greater than 1 indicates antagonism; a CI value equal to 1 indicates an additive effect; and a CI value less than 1 indicates a synergistic effect. In one embodiment, the CI value of a synergistic interaction is less than 0.9. In another embodiment, the CI value is less than 0.8. In another embodiment, the CI value is less than 0.7.

### Glossary

Prior to setting forth this disclosure in more detail, it may be helpful to an understanding thereof to provide definitions of certain terms to be used herein. Unless defined otherwise, all technical and scientific terms used herein have the same meaning as is commonly understood by one of skill in the art to which this invention belongs. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as dose, are to be understood to include any integer within the recited range, unless otherwise indicated. As used herein, the term “about” means ±20% of the indicated range, value, or structure, unless otherwise indicated.

It should be understood that the terms “a” and “an” as used herein refer to “one or more” of the enumerated components. The use of the alternative (e.g., “or”) should be understood to mean either one, both, or any combination thereof of the alternatives.

As used herein, the terms “include,” “have,” and “comprise” are used synonymously, which terms and variants thereof are intended to be construed as non-limiting.

The term “consisting essentially of” limits the scope of a claim to the specified materials or steps, or to those that do not materially affect the basic characteristics of a claimed invention. For example, a protein domain, region, or module (e.g., a binding domain, hinge region, linker module) or a protein (which may have one or more domains, regions, or modules) “consists essentially of” a particular amino acid sequence when the amino acid sequence of a domain, region, or module or protein includes extensions, deletions, mutations, or any combination thereof (e.g., amino acids at the amino- or carboxy-terminus or between domains) that, in combination, contribute to at most 20% (e.g., at most 15%, 10%, 8%, 6%, 5%, 4%, 3%, 2%, or 1%) of the length of a domain, region, or module or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

As used herein, conventional “undetectable” viral load means less than 50 viral copies/mL (<50 cp/mL) as measured by conventionally used assays. For example, viral load may be ascertained by screening, for example, using the Human Immunodeficiency Virus I (HIV-I) Quantitative, RNA assay (Abbott RealTime).

As used herein, an “actual undetectable” viral load means less than or equal to 0 viral copies/mL as measured by a single copy assay.

As used herein, an “extremely low” viral load means less than or equal to 1 viral copy/mL as measured by a single copy assay.

As used herein, a “very low” viral load means less than or equal to 5 viral copies/mL as measured by a single copy assay.

As used herein, a “low” viral load means less than or equal to 10 viral copies/mL as measured by a single copy assay.

As used herein “R5-only tropism” means a cell only susceptible to R5 virus, i.e., by a virus that uses the coreceptor CCR5 for cell entry and infection. R5-only tropism may be ascertained by screening, for example, using a TROFILE® DNA Assay.

As used herein, “conventional complete virologic suppression” means plasma HIV-1 RNA less than 40 copies/mL, which is the lower limit of detection in the commercial assay for HIV detection and is the level at which HIV transmission is reduced by more than 96%.

As used herein, “virologic failure” is defined for purposes of re-initiation of a subject's previous antiretroviral regimen as an HIV-1 RNA level of as two (2) consecutive plasma HIV-1 RNA levels of >400 copies/mL (e.g., Example 1) or, alternatively, as two (2) consecutive plasma HIV-1 RNA levels of >200 copies/mL (Example 2). It is noted that study participants noted in Example 1 having a single plasma HIV-1 RNA level of >400 copies/mL are characterized as not experiencing continued viral suppression, this does not have the same meaning as “virologic failure” as used herein.

As used herein, “chemokine receptor” means a member of a homologous family of seven-transmembrane spanning cell surface proteins that bind chemokines.

As used herein, “CCR5” is a chemokine receptor which binds members of the C—C group of chemokines and whose amino acid sequence comprises that provided in Genbank Accession Number 1705896, and related polymorphic variants.

As used herein, “antibody” means an immunoglobulin molecule comprising two heavy chains and two light chains and that recognizes an antigen. The immunoglobulin molecule may derive from any of the commonly known classes or isotypes, including but not limited to IgA, secretory IgA, IgG, and IgM. IgG subclasses are also well known to those in the art and include but are not limited to human IgG1, IgG2, IgG3, and IgG4. It includes, by way of example, both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. Optionally, an antibody can be labeled with a detectable marker. Detectable markers include, for example, radioactive or fluorescent markers. The antibody may be a human or nonhuman antibody. The nonhuman antibody may be humanized by recombinant methods to reduce its immunogenicity in humans. Methods for humanizing antibodies are known to those skilled in the art.

As used herein, a “small molecule” CCR5 receptor antagonist includes, for example, a small organic molecule which binds to a CCR5 receptor and inhibits the activity of the receptor. In one embodiment, the small molecule has a molecular weight less than 1,500 daltons. In another embodiment, the small molecule has a molecular weight less than 600 daltons. In one embodiment, the small molecule is one or more of maraviroc, vicriviroc, aplaviroc, SCH-C, and TAK-779.

As used herein, “monoclonal antibody,” also designated as “mAb,” is used to describe antibody molecules whose primary sequences are essentially identical and which exhibit the same antigenic specificity. Monoclonal antibodies may be produced by hybridoma, recombinant, transgenic, or other techniques known to one skilled in the art.

As used herein, a “binding fragment” or an “antigen-binding fragment or portion” of an antibody refers to the fragment or portion of an intact antibody that has or retains the ability to bind to the antigen target molecule recognized by the intact antibody, including fragment antigen binding (Fab) fragments, F(ab′)2 fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses genetically engineered or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, tetrabodies, tandem di-scFv, and tandem tri-scFv.

As used herein, “anti-chemokine receptor antibody” means an antibody which recognizes and binds to an epitope on a chemokine receptor. As used herein, “anti-CCR5 antibody” means a monoclonal antibody that recognizes and binds to an epitope on the CCR5 chemokine receptor.

As used herein, “epitope” means a portion of a molecule or molecules that forms a surface for binding antibodies or other compounds. The epitope may comprise contiguous or noncontiguous amino acids, carbohydrate, or other nonpeptidyl moieties or oligomer-specific surfaces.

“Analogs” of antibodies or binding fragments include molecules differing from the antibodies or binding fragments by conservative amino acid substitutions. For purposes of classifying amino acid substitutions as conservative or non-conservative, amino acids may be grouped as follows: Group I (hydrophobic side chains): met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same class. Non-conservative substitutions constitute exchanging a member of one of these classes for a member of another.

As used herein, the term “vector” refers to a nucleic acid molecule that is capable of transporting another nucleic acid. Vectors may be, for example, plasmids, cosmids, viruses, or phage. An “expression vector” is a vector that is capable of directing the expression of a protein encoded by one or more genes carried by the vector when it is present in the appropriate environment.

As used herein, “inhibits” means that the amount is reduced in the presence of a composition as compared with the amount that would occur without the composition.

The term “competitive inhibitor” as used herein refers to a molecule that competes with a reference molecule for binding to a target, and thereby blunts, inhibits, dampens, reduces, or blocks the effects of the reference molecule on the target. For example, PRO 140 is a competitive inhibitor of CCL5 binding to CCR5 receptor.

As used herein, “subject” means any animal, including humans, or artificially modified animal. Artificially modified animals include, but are not limited to, SCID mice with human immune systems. The animals include but are not limited to mice, rats, dogs, guinea pigs, ferrets, rabbits, and primates. In a preferred embodiment, the subject is a human.

As used herein, “treating” means slowing, stopping, or reversing the progression of a given disease or disorder. In a preferred embodiment, “treating” means reversing the progression of the disease or disorder. In some embodiments, treating includes reversing the progression of the disease or disorder to the point of eliminating the disease or disorder.

As used herein, “preventing” refers to preventing a disease or disorder from occurring; delaying the progression of a disease or disorder; stopping the transmission of a disease or disorder to non-infected subjects; or reducing the pathology or symptomatology of a disease or disorder.

As used herein, “administering” may be effected or performed using any of the methods known to one skilled in the art. The methods may comprise oral, intravenous, intramuscular, or subcutaneous means. In a preferred embodiment the mode of administration is by subcutaneous injection.

As used herein, “effective dose” means an amount in sufficient quantities to either treat the subject or prevent the subject from experiencing prolonged uncontrolled HIV-1 viral loads, or to reduce a subject's viral load to one of an undetectable viral load, and actual undetectable viral load, an extremely low viral load, a very low viral load, or a low viral load.

As used herein, a “high dose” or “higher dose” or “high-dose” is any dose of an anti-CCR5 agent greater than conventionally administered amounts that may be used to suppress a subject's viral load count to one of an undetectable viral load, and actual undetectable viral load, an extremely low viral load, a very low viral load, or a low viral load, or at any other specified or target viral load level below 50 viral copies per mL of plasma. In a preferred embodiment, the anti-CCR5 agent is PRO 140 and the high dose is equal to or greater than about 324 mg. For example, a high dose of PRO 140 may be any one of about 350 mg, about 437 mg, about 525 mg, about 700 mg, about 787 mg, etc., or between about 324 mg and 2,000 mg. In a particularly preferred embodiment, a high dose amount of 525 mg of PRO 140 is provided in two 1.5 mL subcutaneous injections, wherein each mL of formulation has a PRO 140 concentration of about 175 mg/mL. In another particularly preferred embodiment, a high dose amount of 700 mg of PRO 140 is provided in two 2 mL subcutaneous injections, wherein each mL of formulation has a PRO 140 concentration of about 175 mg/mL.

Clinical Studies with PRO 140

PRO 140 is a humanized IgG4,_{K} monoclonal antibody (mAb) to the C-C chemokine receptor type 5 (CCR5), under development as a therapy for human immunodeficiency virus (HIV) infection.

PRO 140 binds to the N terminus (Nt) and the extracellular loop 2 (ECL2) domain of the CCR5 cell surface receptor that HIV-1 uses to gain entry to a cell. PRO 140 binding to CCR5 blocks the final phase of viral binding to the cell surface prior to fusion of the viral and cell membranes. PRO 140 has been administered intravenously or subcutaneously to 174 HIV-1 infected individuals in Phase I/II studies of safety, tolerability, pharmacokinetics and pharmacodynamics. Jacobson 2010. The drug has been well tolerated following administration of single doses of 0.5 to 5 mg/kg or up to three weekly doses of up to 324 mg. Single subcutaneous doses of 324 mg have resulted in drops in plasma HIV-1 RNA levels of approximately 1.0 log₁₀. Repetitive weekly administration of this dose of PRO 140 has been associated with drops in plasma HIV-1 RNA levels of approximately 1.5 log₁₀. Serum concentrations of PRO 140 above the IC₅₀ for clinical isolates of HIV-1 are maintained for at least 2 weeks following a single dose of 324 mg. Plasma HIV-1 RNA levels rise to baseline levels as PRO 140 is cleared from the plasma and, presumably, other compartments.

In vitro and in vivo preclinical studies have been conducted to determine the pharmacokinetic, immunogenicity, and toxicity profiles of PRO 140 following IV and SC administration. Several acute and chronic toxicity studies have been conducted to support the clinical development plan.

Acute toxicity of PRO 140 was evaluated in New Zealand rabbits, following IV administration of 5 or 15 mg/kg. Chronic toxicity was evaluated in cynomolgus monkeys following biweekly administration of IV doses up to 10 mg/kg for six months and biweekly administration of various SC doses up to 50 mg/kg for 24 weeks. The drug was generally well tolerated. Biweekly administration of IV doses up to 10 mg/kg for six months resulted in minimum to mild lymphoid hyperplasia in assorted lymph nodes and spleen, which was considered an expected immune response to a foreign protein. Biweekly administration of SC doses up to 50 mg/kg for 24 weeks resulted in minimum injection-site reactions (minimal, multifocal, mononuclear cell infiltrates in the subcutis), which were considered due to an inflammatory response to the injected antigen. Monkeys tolerated treatment with PRO 140 for 24 weeks without evidence of local or systemic toxicity. PRO 140 caused no mortality, cageside observations, in-life injection-site observations, or gross pathologic findings. Chronic treatment with PRO 140 did not affect body weight, food consumption, hematology, clinical chemistry or coagulation parameters.

Both IV and SC administration resulted in elimination half-lives of approximately 200 hours, and overall exposure increased with increasing doses. Following SC administration of PRO 140 in monkeys, the maximal concentration (Cmax) was achieved within 56 hours and bioavailability for PRO 140 after SC dosing was approximately 70%.

Current human experience with PRO 140 consists of seven completed and additional ongoing clinical trials. These studies are summarized in the table below. In all the completed clinical trials, the majority of adverse events (AEs) were mild or moderate. No dose-limiting toxicities or patterns of drug-related toxicities were observed. Antiviral activity was potent, rapid, prolonged, dose-dependent, and highly significant.

TABLE 1-1
Clinical Studies with PRO 140

| | | No. of | | | |
|---|---|---|---|---|---|
| | | Subjects | | | |
| Protocol | | (Planned/ | | Subject | |
| Number | Phase | Analyzed) | Doses | Population | Comments |
| PRO 140 | 1 | 20/20 | Single 0.1, | Healthy | Generally well tolerated; |
| 1101 | | | 0.5, 2.0, or | | non-immunogenic; dose- |
| | | | 5.0 mg/kg | | dependent coating of |
| | | | | | CCR5; significant coating |
| | | | | | of CCR5 over placebo at |
| | | | | | 0.5, 2, and 5 mg/kg |
| PRO 140 | 1 | 20/20 | Either two or | Healthy | Generally well tolerated; |
| 1102 | | | three doses | | SC administration by |
| | | | totaling 200 | | Autoject ® 2 better |
| | | | or 350 mg | | tolerated than manual |
| | | | respectively | | injection |
| PRO 140 | 1 | 15/14 | Two doses, | Healthy | More AEs associated with |
| 1103 | | | each of 350 mg | | arm injection; trend of |
| | | | | | lower exposure in arm |
| | | | | | injections; thigh and |
| | | | | | abdominal administration |
| | | | | | preferred |
| PRO 140 | 1b | 40/39 | Single 0.5, | HIV-1 | Generally well tolerated; |
| 1302 | | | 2.0, or 5.0 mg/kg | positive | antiviral suppression |
| | | | | | maintained for approx. 10 |
| | | | | | days with higher doses; |
| | | | | | favorable tolerability and |
| | | | | | potent, dose-dependent |
| | | | | | antiviral activity provide |
| | | | | | proof-of-concept |
| PRO 140 | 2a | 30/31 | Single 5.0 or | HIV-1 | Generally well tolerated |
| 2301 | | | 10.0 mg/kg | positive | with no dose-limiting |
| | | | | | toxicities; potent antiviral |
| | | | | | suppression maintained for |
| | | | | | approx. 20 days when |
| | | | | | administered IV at 5 or 10 mg/kg. |
| | | | | | No dose-limiting |
| | | | | | toxicities at 10 mg/kg. |
| PRO 140 | 2a | 40/44 | Three doses | HIV-1 | Generally well tolerated, |
| 2101 | | | of 162 or 324 mg | positive | no drug-related SAEs or |
| | | | each | | dose-limiting toxicity; |
| | | | | | antiviral activity was |
| | | | | | statistically significant; |
| | | | | | two-fold exposure at |
| | | | | | higher dose; single dose |
| | | | | | demonstrated favorable |
| | | | | | tolerability, and potent, |
| | | | | | long-acting, dose- |
| | | | | | dependent antiviral |
| | | | | | activity. |
| PRO 140_CD01 | 2b | 43/43 | 350 mg SC | HIV-1 | Generally well tolerated, |
| | | | weekly dose | positive | no drug-related SAEs, |
| | | | for 12 weeks | | weekly dose demonstrated |
| | | | of | | favorable tolerability, and |
| | | | Monotherapy | | potent, long-acting, dose- |
| | | | (total | | dependent antiviral |
| | | | treatment | | activity. |
| | | | duration 14 | | |
| | | | weeks) | | |
| PRO 140_CD01- | 2b | 17/17 | 350 mg SC | HIV-1 | This clinical study is |
| Extension | | | weekly dose | positive | currently ongoing and next |
| | | | for 160 | | protocol amendment to |
| | | | weeks of | | increase the treatment |
| | | | Monotherapy | | duration to 213 weeks is |
| | | | (total | | currently pending |
| | | | treatment | | submission. |
| | | | duration 161 | | |
| | | | weeks) | | |
| PRO 140_CD02 | 2b/3 | 50/52 | Placebo or | HIV-1 | This clinical study is |
| | | | 350 mg SC | positive | currently ongoing. |
| | | | for 1 dose, | | |
| | | | followed by | | |
| | | | 350 mg SC | | |
| | | | weekly dose | | |
| | | | for 24 weeks | | |
| | | | (total | | |
| | | | treatment | | |
| | | | duration 25 | | |
| | | | weeks) | | |

### PRO 140 1302 Study

This initial proof-of-concept study was a randomized, double-blind, placebo-controlled study in subjects with early-stage, asymptomatic HIV infection, only R5 HIV-1 detectable, and no antiretroviral therapy for 12 weeks. Subjects (n=39) were randomized to receive a single IV injection of placebo or PRO 140 at doses of 0.5, 2, or 5 mg/kg. Subjects were monitored for antiviral effects, safety and PRO 140 pharmacokinetics (PK) for 58 days.

The study enrolled 31 males and 8 females. The median age, CD4+ cell count and HIV-1 RNA at baseline were 40.3 years, 484 cells/μL and 26,900 copies/mL, respectively. The baseline characteristics were similar for all treatment groups.

PRO 140 demonstrated potent, rapid, prolonged and dose-dependent antiviral activity. A single 5mg/kg dose reduced viral loads by 1.83 log₁₀ on average. These reductions represent the largest antiviral effects reported after just one dose of any HIV-1 drug. Jacobson 2008. In the 5 mg/kg group, mean viral load reductions of greater than 1 log₁₀ were sustained for 2-3 weeks post-treatment.

There was no change in R5 virus susceptibility to PRO 140 following treatment. All subjects had R5-only virus at screening in the first-generation Trofile assay. R5-only tropism results were observed in all subjects at all other timepoints, with two exceptions: One of nine (11%) of placebo subjects had dual/mixed virus at baseline and all subsequent timepoints, reflecting a spontaneous and stable switch in co-receptor tropism results. One of 30 (3%, 0.5 mg/kg group) had a dual/mixed tropism result on day 8 and R5-only results at all other timepoints, including the end of the day. Jacobson 2008. Clonal analysis of the dual/mixed virus revealed that it reflected outgrowth of pre-existing undetected virus rather than mutation of an R5 virus to a dual/mixed virus following treatment. Marozsan A. J. et al., Clonal analysis of HIV-1 co-receptor tropism change following treatment with PRO 140, a CCR5 monoclonal antibody, 48th Annual ICAAC/IDSA 46th Annual Meeting, Washington, D.C., Vols. Abstract H-1218 (2008). Therefore, no significant development of viral resistance to PRO 140 was observed despite potent and prolonged (2-3 weeks on average) viral suppression, followed by slow washout of the drug. Given that resistance to other classes of HIV-1 drugs can develop within one week of monotherapy, the findings indicate that PRO 140 presents a high barrier to viral resistance in vivo. Demeter L M et al., Delavirdine susceptibilities and associated reverse transcriptase mutations in human immunodeficiency virus type 1 isolates from patients in a phase I/II trial of delavirdine monotherapy (ACTG 260), ANTIMICROB.AGENTS CHEMOTHER., Vol. 44, pp. 794-797 (2000); Saag M. S. et al., A short-term clinical evaluation of L-697,661, a non-nucleoside inhibitor of HIV-1 reverse transcriptase, N. ENGL. J. MED., Vol. 329, pp. 1065-1072 (1993); Richman D. D. et al., Nevirapine resistance mutations of human immunodeficiency virus type 1 selected during therapy, J. VIROL., Vol. 68, pp. 1660-1666 (1994).

Serum levels increased with increasing dose. The mean Area Under Curve (AUC) from time zero to infinity (AUC∞) values were 11.1, 74.3 and 278 mg×day/L for the 0.5, 2 and 5 mg/kg groups. The mean serum half-life was 3.5-3.9 days in the two highest dose groups. In addition, PRO 140 significantly masked CCR5 on circulating lymphocytes for 2-4 weeks. Jacobson 2008. The PK and receptor occupancy data were broadly consistent with the duration of antiviral effects. The mean serum half-lives were 3.9 days and 3.5 days in the 2 mg/kg and 5 mg/kg dose groups, respectively.

Intravenous PRO 140 was generally well tolerated. No drug-related serious events or dose-limiting toxicity was observed. The most common adverse events (headache, lymphadenopathy, diarrhea, and fatigue) were observed at similar frequencies across the placebo and PRO 140 dose groups. There was no significant effect on QTc interval intervals or other electrocardiographic parameters, and there were no remarkably laboratory findings. There was no loss or depletion of CD4+ or CCR5 + cells from the circulation. At the 5 mg/kg dose, there was a trend towards increased CD4+ cell counts from baseline, with mean changes of +129, +96 and +83 cells/μL observed on days 8, 15, and 22, respectively.

### PRO 140 2301 Study

PRO 140 2301 was a multi-center, randomized, double-blind, placebo-controlled, parallel group study in 30 male and female adult subjects infected with HIV-1. Subjects were randomized to one of three groups (N=10/group), each receiving one of three treatments: (i) a single IV dose of 5 mg/kg by 30-minute IV infusion; (ii) a single IV dose of 10 mg/kg by 30-minute IV infusion; (iii) a single placebo dose by 30-minute IV infusion. The objective of the study was to assess and characterize the PK and PD of PRO 140 administered by IV infusion, assess efficacy at a new dosage level, and safety and tolerability of single doses of PRO 140.

All PRO 140-treated subjects had more than 10-fold reduction in viral loads (mean max logᵢₙ reductions were 1.83 for treatment groups and 0.32 for placebo). Both the 5 mg/kg and 10 mg/kg doses have shown favorable tolerability and no dose-limiting toxicity has been observed. High levels of receptor occupancy (>85% reduction in the number of cells detected) were observed for 29 days after treatment with both 5 and 10 mg/kg doses.

PRO 140 is a humanized monoclonal antibody targeting CCR5 with potent antiviral activity in patients with CCR5 -tropic HIV-1 infection. In phase 2b studies, the long-term efficacy, safety, and tolerability of PRO 140 monotherapy in maintaining viral suppression for over 24 months was evaluated in patients who were stable on combination antiretroviral therapy on entry into the trials. These studies are summarized here and also reported in Dhody et al., PRO 140, a monoclonal antibody targeting CCR5, as a long-acting, single-agent maintenance therapy for HIV-1 infection, HIV CLINICAL TRIALS, vol. 19, no. 3 (2018).

### EXAMPLE 1

### HIV-1 Infected Subject Viral Load Suppression After Four Weeks PRO 140 SC Monotherapy Relative to Single Copy RNA Viral Load Count Prior to Monotherapy

Interim data from subjects where PRO 140 was administered as a 350 mg subcutaneous (SC) injection weekly was analyzed to look at the importance of viral load counts, including viral load counts below 40 copies/mL or 50 copies/mL, as such may relate to monotherapy success. PRO 140 350 mg subcutaneous injection was administered to subjects in two consecutive doses and study participants were monitored for viral rebound on a weekly basis.

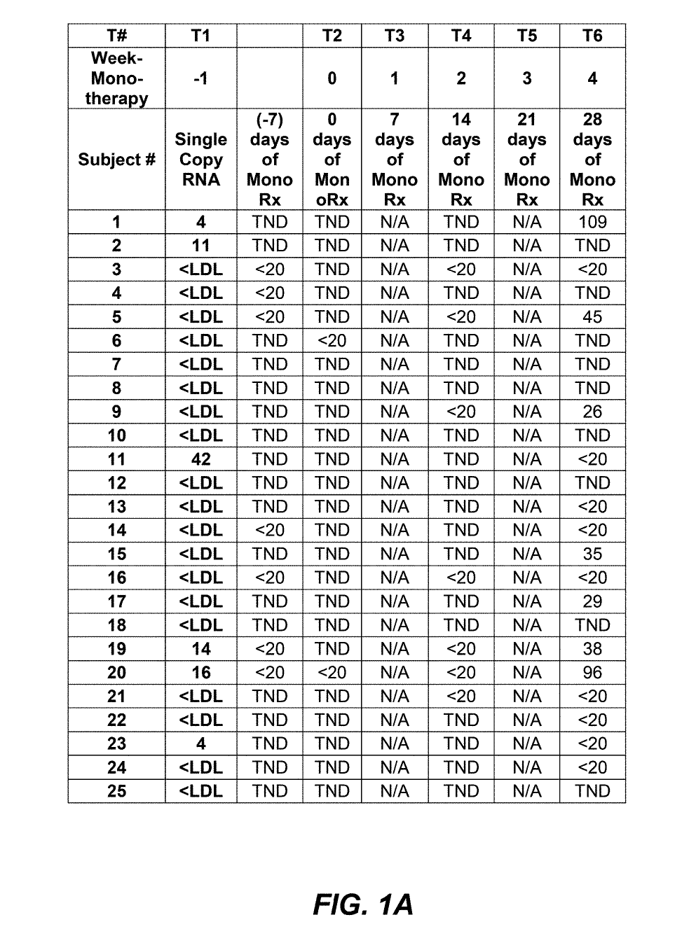

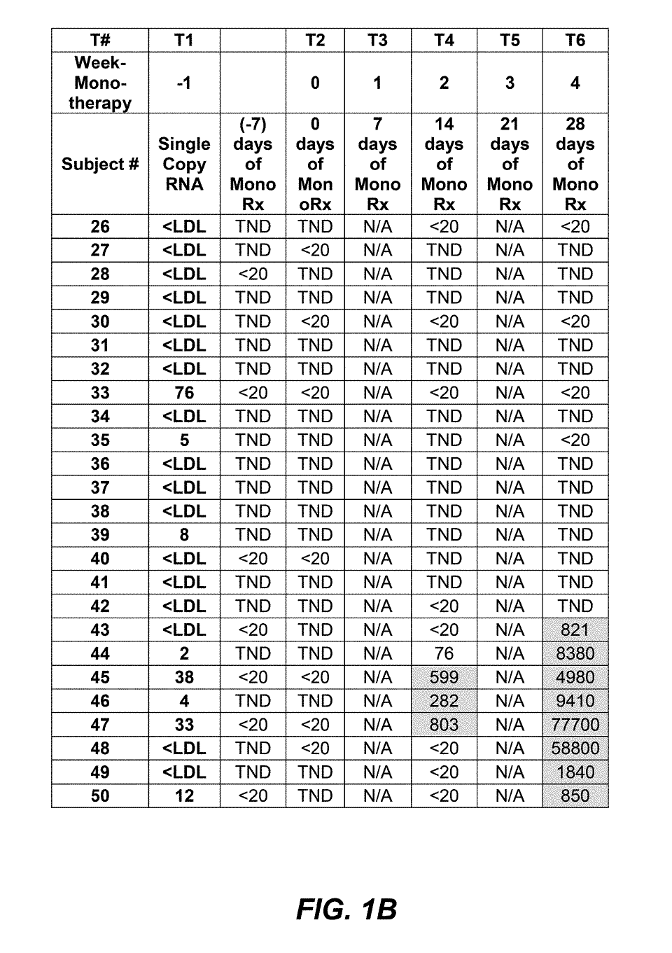

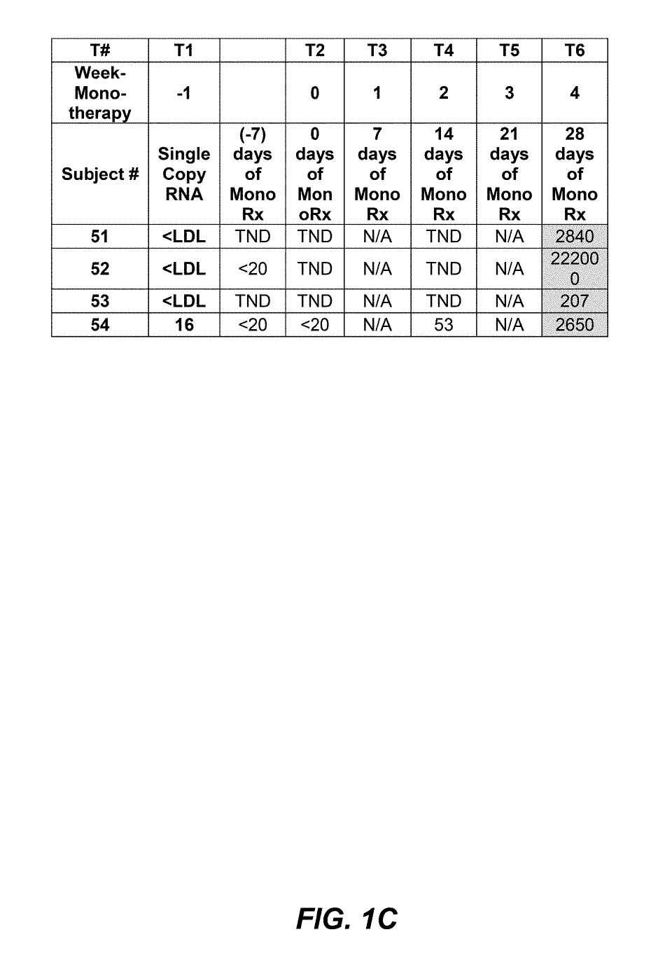

An interim data set for fifty-four (54) subjects having completed four (4) weeks of SC PRO 140 monotherapy is provided in FIG. 1A, FIG. 1B, and FIG. 1C. As shown in FIG. 1A, FIG. 1B, and FIG. 1C, of these fifty-four (54) subjects, forty-two (42) maintained viral load levels that were actually undetectable, or totally non-detectable (TND), <20 copies/mL, or less than 109 copies/mL at four (4) weeks. Twelve (12) of the fifty-four (54) subjects had a viral load >400 copies/mL at four (4) weeks.

It is understood that unrelated viral infections, such as common colds, etc., may trigger impermanent bumps or rises in detected viral load such that, assuming a subject has exclusively R5-tropic virus they may carry a viral load that temporarily deviates but ultimately returns to a successfully suppressed viral load.

Here, forty-two out of fifty-four (42/54) subjects, or 77.8% of the subjects experienced continued viral suppression after four (4) weeks of subcutaneous PRO 140 monotherapy. Twelve out of fifty-four (12/54) subjects, or 22.2% of the subjects experienced viral load counts greater than 400 copies/mL after four (4) weeks of SC PRO 140 monotherapy.

Of the forty-two (42) subjects, after four (4) weeks of subcutaneous PRO 140 monotherapy, twenty-two (22) had no actual detectable or extremely low viral load, thirteen (13) had a viral load count of <20 copies/mL, and the remaining seven (7) subjects had a viral load of less than or equal to 109 copies/mL. Of the forty-two (42) subjects, after four (4) weeks of SC PRO 140 monotherapy, thirty-three (33) had no actual detectable or extremely low viral load upon initiation of subcutaneous PRO 140 monotherapy.

Of the twenty-two (22) subjects having no actual detectable viral load after four (4) weeks of subcutaneous PRO 140 monotherapy, twenty (20) of these subjects (20/22, or 90.9%) also had no actual detectable or extremely low viral load prior to initiation of SC PRO 140 monotherapy. This indicates that a strong indicator of success of subcutaneous PRO 140 monotherapy may be having no actual detectable or extremely low viral load prior to initiation of subcutaneous PRO 140 monotherapy. However, of the twelve (12) subjects who did not experience continued viral suppression after four (4) weeks of SC PRO 140 monotherapy, six (6) also had no actual detectable or extremely low viral load prior to initiation of SC PRO 140 monotherapy.

Out of the fifty-four (54) subjects, thirty-nine (39) had no actual detectable or extremely low viral load prior to initiation of SC PRO 140 monotherapy, and twenty (20) of these subjects went on to have no actual detectable or extremely low detectable viral load after four (4) weeks of SC PRO 140 monotherapy. That is, 20/39, or 51.2%, of those subjects having no actual detectable or extremely low viral load prior to initiation of subcutaneous PRO 140 monotherapy continued to have no actual detectable or extremely low viral load after four (4) weeks of subcutaneous PRO 140 monotherapy.

Here, two (2) additional subjects achieved no actual detectable or extremely low viral load after four (4) weeks of subcutaneous PRO 140 monotherapy by using criteria that expanded the eligibility pool from those thirty-nine (39) subject that had no actual detectable or extremely low viral load prior to initiation of subcutaneous PRO 140 monotherapy to include an additional fifteen (15) subjects having a detectable viral load count <50 copies/mL but greater than no actual detectable or extremely low viral load prior to initiation of subcutaneous PRO 140 monotherapy. Compared to the 51.2% (20/39) of those subjects having no actual detectable or extremely low viral load prior to initiation of subcutaneous PRO 140 monotherapy that continued to have no actual detectable or extremely low viral load after four (4) weeks of SC PRO 140 monotherapy, only 13.3% (2/15) of those subjects having a detectable viral load <50 copies/mL but greater than no actual detectable or extremely low viral load prior to initiation of SC PRO 140 monotherapy had no actual detectable or extremely low viral load after four (4) weeks of SC PRO 140 monotherapy.

In view of these interim results, the impact of permitting broader eligibility criteria for consideration of SC PRO 140 monotherapy is, thus, considered anew. For the first time, the present inventors find reason to question adherence to the conventional understanding of “undetectable” viral load as 50 copies/mL or of complete virologic suppression defined as plasma HIV-1 RNA less than 40 copies/mL. This is because, upon closer inspection of no actual detectable or extremely low viral load counts versus other detectable viral loads <50 copies/mL, important and significant differences appear to exist in the prognosis of how different subjects will respond to therapeutic treatment, including subcutaneous PRO 140 monotherapy.

Importantly, as shown here, subjects having no actual detectable or extremely low viral load prior to initiation of subcutaneous PRO 140 monotherapy may be about 3.85 times (51.2% (20/39)/13.3% (2/15)=3.85 times), or about four times, more likely to have no actual detectable or extremely low viral load after four (4) weeks of subcutaneous PRO 140 monotherapy than are those subject with a detectable viral load <50 copies/mL but greater than no actual detectable or extremely low viral load. Thus, this interim data suggests that, even among subjects conventionally understood to have undetectable viral loads that are seeking to achieve no actual detectable or extremely low viral load after four (4) weeks of subcutaneous PRO 140 monotherapy, such subjects are about four (4) times more likely to achieve success if they have no actual detectable or extremely low viral load prior to initiation of subcutaneous PRO 140 monotherapy. Further, this interim data suggests that, even among subjects conventionally understood to have undetectable viral loads that are seeking to achieve continued or prolonged success using subcutaneous PRO 140 monotherapy, such subjects are more likely to achieve success if they have no actual detectable or extremely low viral load prior to initiation of subcutaneous PRO 140 monotherapy.

Accordingly, this information may have great import for patients and physicians alike when considering therapeutic options to achieve prolonged viral suppression, viral suppression to no actual detectable or extremely low viral load levels, and likelihood of monotherapy success. Improved patient selection to identify potentially responding patients may further justify use of PRO 140 as a simplified maintenance monotherapy for HIV-1 infected subjects. Further consideration of alternative options to expand the potential patient pool by achieving viral suppression to no actual detectable or extremely low viral load levels prior to initiation of monotherapy for HIV-1 infected subjects is also justified. Improved patient selection to identify potentially responding patients, or promotion to render additional patients well-suited for PRO 140 monotherapy, may further justify use of PRO 140 as a simplified maintenance monotherapy for HIV-1 infected subjects.

### EXAMPLE 2

### Rationale for Dose Selection

The dose of 350 mg administered SC administered, for example, in Example 1, was chosen in light of a previous analysis suggesting that such a dose would be likely to provide maximal viral load suppression.

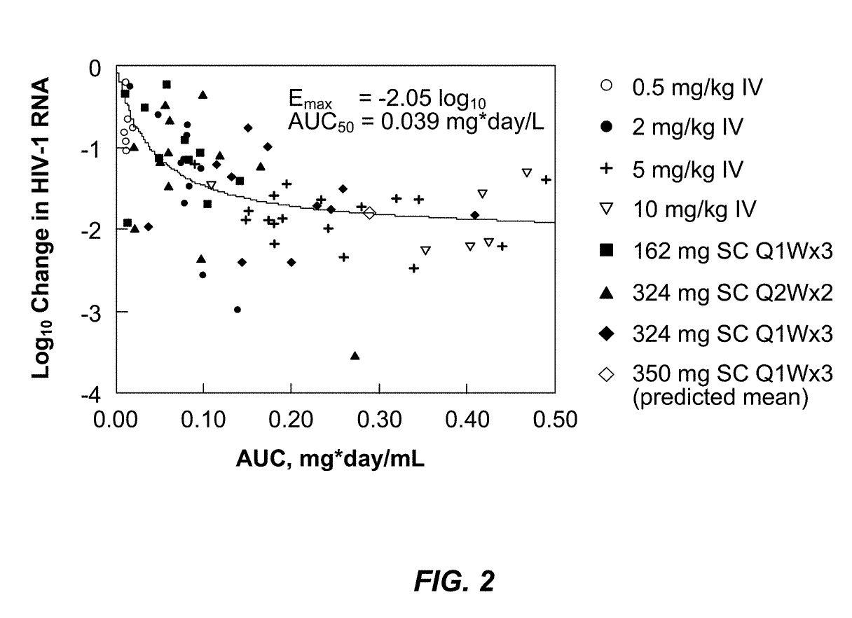

In studies with antiviral agents that block viral entry through the CCR5 receptor, it is conventionally believed that in order to achieve robust antiviral effects and minimize the potential for drug resistance in combination therapy, the dose of drug should result in exposures that fall on the plateau of a Maximum Drug Effect (Emax) plot. FIG. 2 shows an Emax analysis of antiviral data generated with IV and SC PRO 140.

The maximal viral load reduction was analyzed with regard to drug exposure for PRO 140. FIG. 2 shows this relationship. Analysis shows that PRO 140 350 mg weekly dose is expected to fall on the plateau of the Emax plot. Here, the maximal change in HIV-1 viral load from baseline was determined at any point 59 days after initiation of therapy. To allow approximate comparisons between the IV and SC doses, the overall AUC observed for repeat SC doses was conservatively estimated by multiplying the measured AUCO-7d by the number of doses administered. Viral load and AUC data were fit to an Emax equation: E=Emax×AUC/(AUC+AUC50). The black diamond outline indicates projected data for three weekly 350 mg doses based on the mean exposure observed in the PRO 140 1103 study.

It is important to note that when larger proteins (MW>10,000) are administered SC, they initially traffic through the lymphatic system. Uptake into the bloodstream occurs after the proteins reach the thoracic duct. Nishikawa M et al., Analysis of binding sites for the new small-molecule CCR5 antagonist TAK-220 on human CCR5, ANTIMICROB. AGENTS CHEMOTHER., Vol. 49 (11), pp. 4708-4715 (2005). In addition, based on pharmacodynamic data from our prior SC and IV studies, maximum virologic suppression is expected to be achieved with trough concentrations that equal or exceed approximately 5 μg/mL.

Finally, the mean nadir reduction in viral load achieved with 3 weekly 324 mg SC doses (1.65 log₁₀) was similar to the mean nadir reductions observed with single 5 or 10 mg/kg IV doses (1.8 log₁₀ in each case). Overall, several lines of evidence indicate that maximum virologic suppression will be achieved with 350 mg weekly dosing.

While a 350 mg dose amount is supported by conventional approaches for such dosage amount determinations and that this dosage was effective in a monotherapy setting for a certain subset of patients, it is noted that about half of subjects receiving 350 mg weekly SC dosing in a monotherapy setting experienced virologic failure in CD01-Extension study. Importantly, however, review of available PRO 140 clinical data with 350 mg SC weekly dosing, suggested no evidence of emergence of viral isolates with reduced susceptibility to PRO 140, no altered viral tropism or anti-PRO 140 antibodies formation. Accordingly, this review of the PRO 140 clinical data suggested to the inventor that the most likely cause of viral rebound is inadequate dosing to fully cover CCR5 receptor populations. Based on pharmacologic modeling studies, the inventor expects that a 525 mg and 700 mg dose will result in a lower fraction of study participants with trough levels below that which will ‘uncoat’ a significant number of CD4 cells (i.e., less than a certain multiple of the IC₅₀ or IC₉₀ for PRO 140).

### EXAMPLE 3

### PRO 140, a Monoclonal Antibody Targeting CCR5, as a Long-Acting, Single-Agent Maintenance Therapy for HIV-1 Infection

Forty-one adult patients, infected exclusively with CCR5-tropic HIV-1 with viral loads <50 copies/mL, were switched from daily oral combination ART regimens to weekly PRO 140 monotherapy for 12 weeks. Participants who completed 12 weeks of treatment without experiencing virologic rebound were allowed to self-administer PRO 140 as a 350 mg subcutaneous injection weekly, for up to an additional 160 weeks. These studies are summarized here and also reported in Dhody et al., PRO 140, a monoclonal antibody targeting CCR5, as a long-acting, single-agent maintenance therapy for HIV-1 infection, HIV CLINICAL TRIALS, vol. 19, no. 3 (2018).

Participants were monitored bi-weekly for one year, and every four weeks thereafter for virologic rebound. PRO 140 provided virologic suppression in 23/41 (56.1%) participants for 12 weeks and was well tolerated. Ten (10) participants continued and at least nine participants have completed more than two years of monotherapy treatment (47-129 weeks). At the two-year time point, seven of the 10 study participants had viral loads of less than 1 copy/mL using single-copy HIV RNA assay (bioMONTR lab), while the other three had values of 4, 10, and 19 copies/mL. Participants experiencing virologic rebound achieved full viral suppression upon re-initiation of oral combination ART regimen. Anti-PRO 140 antibodies were not detected in any patient, and no drug-related major adverse events or treatment discontinuations were reported.

The Phase 2b study (CD01) was designed to evaluate the efficacy, safety, and tolerability of PRO 140 monotherapy for the maintenance of viral suppression in participants who were stable on antiretroviral therapy (ART). The study protocol required participants to have a plasma HIV-1 viral load less than 50 copies/mL, CD4 cell count greater than 350/mm³, exclusive CCR5-tropic virus, on stable highly active ART (HAART) for 12 months with no change in regimen four weeks prior to screening, and no prior use of maraviroc. The median duration of prior ART regimen was five years. All enrolled subjects were on a combination of three or more ART drugs in which 17 had integrase inhibitors, 15 had NNRTI, and had protease inhibitors (nine boosted and two unboosted) as their third drug in the baseline ART regimen. These regimens were stopped at the start of treatment with PRO 140 monotherapy.

It is known that if viral rebound occurs while NNRTI levels are at sub-therapeutic levels (when HAART is stopped), NNRTI resistance may emerge. To avoid the possibility of viral rebound, there was a one week overlap of existing retroviral regimen and PRO 140 at the beginning of the study treatment built into the study to avoid the emergence of NNRTI resistance by “covering the NNRTI tail.”

HIV-1 co-receptor tropism was evaluated at the screening visit using the Trofile® DNA Assay performed at Monogram Biosciences (South San Francisco, Calif.). Study participants were shifted from daily oral ART to 350 mg PRO 140 monotherapy for up to 12 weeks. PRO 140 was administered by a qualified medical professional or by self-administration. Subjects choosing to self-administer PRO 140 were trained by a licensed medical professional (MD, DO, PA, LPN, LVN, NP, or RN) at the site. The subject was then to self-administer PRO 140 under direction observation of the aforementioned site personnel. Subjects who were able to successfully self-administer the study treatment multiple times, per the site personnel's discretion at the clinic, were then given a supply of PRO 140 as well as a self-administration instruction sheet for the subsequent visits. Study participants were monitored for viral rebound on a weekly basis following initiation of PRO 140 monotherapy and re-initiated their previous antiretroviral regimen if plasma HIV-1 RNA levels rose above 400 copies/mL on two consecutive blood draws at least three days apart.

Study participants were monitored for viral rebound on a weekly basis following initiation of PRO 140 monotherapy and re-initiated their previous antiretroviral regimen if plasma HIV-1 RNA levels rose above 400 copies/mL on two consecutive blood draws at least three days apart.

Participants that experienced virologic rebound moved to the Follow-up Phase, restarted oral ART, and were monitored every four weeks for plasma HIV-1 RNA and CD4 T-cell count until viral load returned to less than 50 copies/mL. These participants were followed for up to 24-36 months after re-initiation of baseline ART to assess the durability of viral suppression after exposure to PRO 140 monotherapy.

The study initially enrolled 40 participants across two separate cohorts, with 12 participants enrolled under Cohort 1 and 28 participants enrolled in Cohort 2 after a DSMB evaluation of safety and efficacy data from Cohort 1. A third Cohort was added after the enrollment of 40 participants was completed. Sixty-eight (68) additional 6 patients screened for Cohort 3. Subjects in Cohorts 2 and 3 that completed 12 weeks of treatment under the CD01 protocol without experiencing virologic rebound could enter the Phase 2b Extension Study, which was designed to evaluate the long-term efficacy, safety, and tolerability of PRO 140 monotherapy for the maintenance of viral suppression. Eligible participants continued PRO 140 monotherapy for up to an additional 160 weeks under a study extension protocol. Drug concentration was assessed through analysis of population PK. The blood samples for PK measurements were taken every four weeks starting from the baseline visit (prior to initiation of PRO 140 monotherapy). The blood samples were collected at the end of the dosage interval (trough level) i.e. prior to the subsequent PRO 140 dosing.

This was an open-label study performed at a single center in San Francisco (Calif.), with eligible participants identified through referrals and site database. The primary efficacy endpoint of the CD01 and the CD01 extension studies was time to loss of virologic response after initiating PRO 140 monotherapy. Secondary endpoints evaluated the number of participants with virologic rebound at the end of the Treatment Phase, as well as mean change in viral load and CD4 cell count across the Treatment Phase.

In the CD01 study, HIV-1 RNA was evaluated weekly using a quantitative assay (Abbott Real Time) with a lower limit of detection of 40 copies/mL. The CD4 cell count was assessed weekly for Cohort 1, and biweekly for Cohorts 2 and 3 using a TruCount Assay (LabCorp). In the CD01 Extension study, HIV-1 RNA and CD4 T-cell count (LabCorp) monitoring was done bi-weekly from weeks 12 to 52, then once every four weeks thereafter. Single-copy HIV RNA levels (bioMONTR Lab) were also evaluated at the two-year time point.

Cellular HIV DNA from all enrolled participants who experienced virologic rebound was tested for viral tropism phenotype using the PhenoSense® Entry Assay (Monogram Biosciences). HIV-1 RNA from plasma viral RNA obtained at the time of virologic rebound was used to construct envelope recombinant viruses. The ability of test compounds, AMD3100, maraviroc, and PRO 140, to block entry of recombinant viruses bearing these envelopes into CD4 T-cells expressing either the CCR5 or the CXCR4 receptor was assessed and compared to the concentrations required to block similar recombinant viruses constructed from pre-treatment cellular HIV DNA sequences in order to assess changes in 50 and 90% Inhibitory Concentrations (IC50 and IC90) during the course of the study.

Participants were assessed for the development of anti-idiotypic antibodies and the pharmacokinetic properties of PRO 140 (QPS, LLC). In the CD01 study, samples were taken at the Screening Visit, Treatment Visits 4, 8, and 12, and at virologic rebound, as well as the second week or fourth week of the Follow-up Phase. In the CD01 Extension study, all participants had laboratory samples collected at Screening Visit 1, at every fourth treatment visit, and if applicable at virologic rebound. There was no correlation between higher PRO 140 concentrations and adverse events.

Serum concentration of ART drugs was determined during the treatment phase in both studies to confirm adherence to the monotherapy regimen (Consolidated Laboratory Services, LLC).

Safety was assessed by the evaluation of tolerability of repeated SC administration of PRO 140, as assessed by study participants (using Visual Analog Scale), investigator evaluation of injection site reactions, frequency of Grade 3 or 4 adverse events as defined by the DAIDS Adverse Event scale, and frequency of treatment—emergent serious adverse events.

Data analyses were performed with SAS® software, version 9.3. All data collected from the two studies were presented as by-participant listings and also summarized according to the variable type. Summary statistics for continuous variables were presented using number of observations, mean, median, range, and standard deviation. Summary statistics for categorical variables were presented as frequency count and percentage. There were no pre-planned analyses of covariates and no imputation of missing data was performed.

Results. Forty-three (43) participants (Male/Female: 38/3) with median age of 55 years (26-72), median time since HIV diagnosis of 19 years (2-37) and median CD4 T-cell count of 609 cells/mm3 (365-1240) were enrolled in the CD01 study. Two (2) patients were deemed ineligible for efficacy analysis post-enrollment due to presence of dual/mixed tropic virus in a blood sample collected at Screening/Baseline. Sixteen (16) eligible participants, 14 male and two female, with a median age of 54.5 years (26-67) were enrolled in the CD01 Extension study. The majority of participants were Caucasian (81.3%). Participants had a median time since HIV diagnosis of 12.5 years (2-37) and median CD4 cell count of 593 cells/mm3 (365-1059). In addition, the majority of subjects enrolled elected to self-administer PRO 140 in the extension protocol.

Efficacy. In both studies, the primary efficacy endpoint was time to loss of virologic response after initiating PRO 140 monotherapy. Twenty-three (23) of 41 participants (56.1%) in the CD01 study maintained viral suppression throughout the 12 week monotherapy treatment phase. Seven (7) of these participants completed one week overlap of oral ART and PRO 140 at the end of Treatment Phase and moved into the Follow-up Phase, while the other sixteen (16) participants continued PRO 140 monotherapy in the ongoing CD01 Extension study.

Eighteen (18) subjects did not maintain viral suppression during the 12 week monotherapy treatment phase in the CD01 study. The mean time to virologic rebound was 51.3 days, ranging from 28 to 78 days. Participants who experienced virologic rebound moved to Follow-up Phase and restarted oral combination ART. Once ART was reinitiated, all 18 virologic rebound patients achieved viral suppression to less than 50 HIV-1 RNA copies/mL, with mean time to viral suppression of 46.6 days.

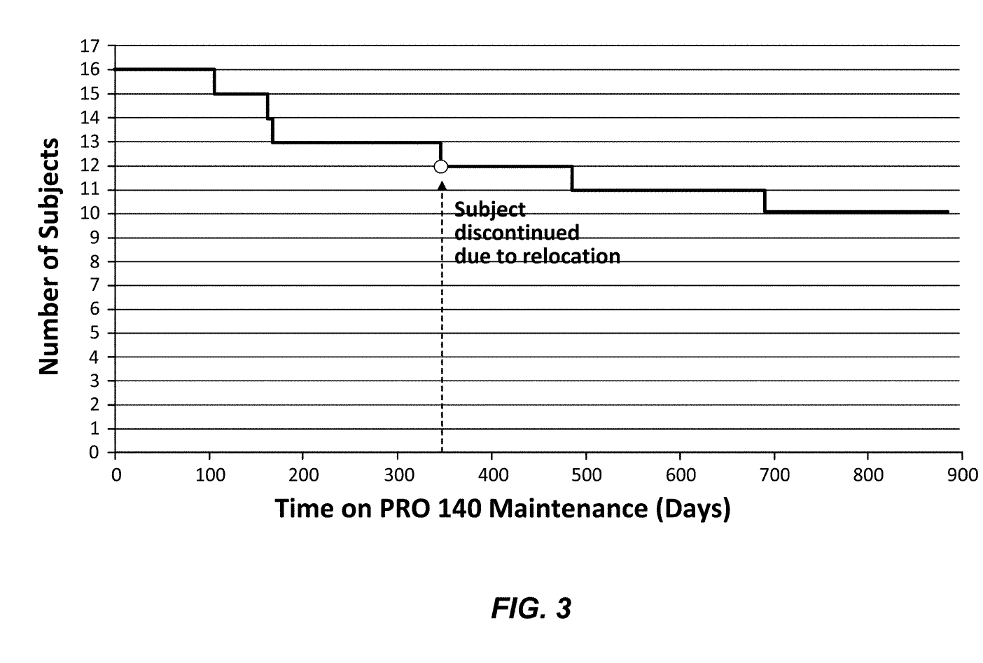

In the CD01 Extension study, 10 of the 16 participants remain in the study, of whom nine have completed over two years of treatment (FIG. 3). One patient discontinued due to relocation after 49 weeks of virologic suppression and five participants experienced virologic rebound. The mean time to virologic rebound was 323 days. Participants unable to maintain viral suppression on PRO 140 monotherapy had their baseline ART regimen re-initiated, and all achieved complete viral suppression after ART re-initiation.

Participants experiencing virologic rebound were followed for up to 24 to 36 months after re-initiation of baseline ART and showed no long-term virologic or clinical consequences as a result of rebounding on PRO 140 monotherapy. The 10 participants currently ongoing in the CD01 Extension study have received PRO 140 monotherapy for time periods ranging from 47 to 129 weeks. Nine (9) of the 10 participants have completed more than two years of treatment with PRO 140 monotherapy. HIV-1 RNA levels remained suppressed below 40 copies/mL for 81% (13/16) of participants for greater than 40 weeks and greater than two years for 62.5% (10/16) of participants.

At the two-year time point, seven of the 10 study participants had viral loads of less than 1 copy/mL using single-copy HIV RNA assay (bioMONTR lab), while the other three had values of 4, 10, and 19 copies/mL. In the CD01 Extension study, each patient demonstrated only CCR5-tropic HIV-1 virus at Screening, and no change in co-receptor tropism was reported when reassessed at virologic rebound.

Individual patient analysis of IC₅₀ and IC₉₀ values showed no significant changes in post-treatment values compared with pre-treatment baseline values for three test compounds, PRO 140, maraviroc, and AMD3100 in either the virologic rebound or non-virologic rebound groups. However, an aggregate analysis showed that the participants which experienced virologic rebound had higher IC₉₀ values for PRO 140 at baseline (10.8 μg/mL) compared to participants without virologic rebound (6.7 μg/ mL).

Anti-PRO 140 antibodies were not detected in any post-treatment sample from either study. The serum concentration (mean +/−SD) of PRO 140 at 4, 8, and 12 weeks of treatment was 18.2+/−8.5, 22.1+/−8.9, and 24.6+/−13.5 ug/mL, respectively. PRO 140 had a PK profile similar to that seen in prior clinical studies.

Safety. Safety data were analyzed for 41 participants in the CD01 study and 16 participants in the CD01 Extension study (Table 1). One of 41 participants in the CD01 study experienced a serious adverse event (SAE), reported by MedDRA preferred term as transient ischemic attack, which was deemed not related to the study drug by the Principal Investigator. One of 16 participants in the CD01 Extension study experienced a SAE, reported by MedDRA preferred term as a bile duct stone, which was deemed not related to the study drug by the Principal Investigator.

In both studies, all definitely and probably treatment-related AEs were local injection site reactions and were mild, transient, and self-resolving. No other clinically relevant treatment-related effects were observed. The incidence of clinically notable abnormalities in vital signs, physical examination, and clinical laboratory tests was low.

Discussion. In the CD01 proof of concept PRO 140 monotherapy study, more than half of participants maintained viral suppression over the duration of 12 weeks, indicating the potential of PRO 140 to maintain viral suppression in a certain population of HIV patients. Virologic rebound patients achieved viral re-suppression after re-initiation of baseline ART regimen. Participants experiencing virologic rebound were followed for up to 36 months after re-initiation of baseline ART and showed no long-term virologic consequences as a result of PRO 140 monotherapy.

In the ongoing, proof of concept, long-term CD01 Extension study, 10 of the 16 eligible participants remain in the study, having received PRO 140 monotherapy for time periods ranging from 47 to 129 weeks. Sustained antiviral activity of PRO 140 was demonstrated with HIV-1 RNA levels continually suppressed for greater than two years for 62.5% (10/16) of participants. It should be pointed out that on an intent-to-treat basis, which includes both studies, the percent of patients without viral rebound would only be 33% (10/30). The single copy HIV-1 RNA assay showed viral suppression of less than 1 copy/mL in 70% (7/10) of participants at the two-year time point. With improved patient selection to identify potentially responding patients, further development of PRO 140 as a simplified maintenance monotherapy regimen for HIV-1 infection could be justified.

Overall, PRO 140 was well tolerated with no related SAEs or discontinuation due to AEs observed in these studies. Other potential benefits of PRO 140 monotherapy include reductions in ART non-adherence and toxicity, along with reduction in other complaints related to intolerance of combination ART regimens.

Given the limited sensitivity and specificity of the Trofile® DNA Assay, it was not unexpected that two participants were reported as having dual/mixed (D/M) tropism at the time of virologic rebound using the standard Trofile® RNA Assay. The emergence of CXCR4-tropic virus was likely due to pre-existing CXCR4-tropic viruses rather than true co-receptor “switching,” as no phenotypic shift in the IC50 and IC90 concentration was observed.

There was no significant change in viral susceptibility to PRO 140 in virologic rebound and non-virologic rebound groups of patients assessed by post-treatment IC₅₀, and IC₉₀ values when compared with pretreatment baseline values. This indicates that the ligand-receptor recognition profile of the CCR5 co-receptor was not altered during the course of the study. In addition, no changes in HIV-1 co-receptor tropism following virologic rebound were seen. PhenoSense® Entry results for PRO 140, maraviroc, and AMD3100 showed no significant change in post-treatment IC₅₀, and IC₉₀, compared with baseline results in virologic rebound patients. However, there was a noted difference in the IC₉₀ values from virologic rebound (10.8+/−9.28) and non virologic rebound (6.7+/−6.8) groups on entry analysis indicating that more PRO 140 was required to reach IC₉₀ by the group that was destined to rebound on PRO 140 monotherapy.

In the absence of evidence of emergence of viral isolates with reduced susceptibility to PRO 140, altered viral tropism or anti-idiotypic PRO 140 antibodies, the cause of viral rebound is yet to be resolved. The determination of methods to select patients that may respond to PRO 140 monotherapy is clearly needed. Limitations of the CD01 study include the high variability in the duration of HIV diagnosis, the extent of prior ART exposure in participants enrolled, the lack of baseline antiviral genotypic and/or phenotypic drug resistance profile for patients enrolled in this study. The ongoing CD01 Extension study is limited by population size, though the results show PRO 140 monotherapy has maintained HIV-1 RNA levels below 40 copies/mL for more than 3 years, and has exhibited an excellent long-term safety profile.

It is notable that other monotherapy strategies with protease inhibitors and recently with dolutegravir have failed. Paton N I et al., Protease inhibitor monotherapy for long-term management of HIV infection: a randomised, controlled, open-label, non-inferiority trial, LANCET HIV. 2(10):e417-e426 (2015); Wijting I et al., Dolutegravir as maintenance monotherapy for HIV-1: a randomized clinical trial, Program and abstracts of the 2017 CONFERENCE ON RETROVIRUSES AND OPPORTUNISTIC INFECTIONS, Feb. 13-16, 2017; Seattle, Wash. Abstract 451LB; Blanco I L et al., Pathways of resistance in subjects failing dolutegravir monotherapy, Program and abstracts of the 2017 Conference on Retroviruses and Opportunistic Infections; Feb. 13-16, 2017, Seattle, Wash. Abstract 42. This is further evidence that monotherapy in general is difficult and may be particularly so with agents directed at inhibiting the viral life cycle internally rather than entry inhibitors.

PRO 140 has a potential to address an unmet need for a simplified, long-acting, single-agent, maintenance regimen for HIV infection if host and/or virologic factors that predict treatment success on PRO 140 monotherapy can be identified. Currently, a large, multi-center, investigative Phase 2b/3 clinical study is underway to determine the cause for virologic rebound observed in the CD01 and CD01 Extension studies.

In summary, this trial showed that PRO 140 was potent enough and well enough tolerated that a substantial fraction of people could be suppressed on it alone for over three years. Over that time, there were no non-injection site AEs, no anti-PRO 140 antibodies detected, no selection of X4 virus, and even those who failed could universally be re-suppressed by returning to their original regimens. The fact that a good fraction of the participants could be suppressed so well with PRO 140 monotherapy for now over three years is strong support of the concept that this agent can become an important component of a long-acting combination regimen in this era when there is intensified interest in this approach for prevention and therapy. It could also be combined with multiple other agents including other monoclonals such as ibalizumab, broadly neutralizing anti-HIV antibodies and nano-formulated small molecules like cabotegravir and rilpivarine.

Conclusion. PRO 140 has a potential to address an unmet need for a long-acting, single-agent, maintenance regimen for HIV infection in selected patients. Studies are underway to determine host and/or virologic factors that may predict treatment success on PRO 140 monotherapy. Moreover, PRO 140 has sufficient potency for a prolonged period of monotherapy such that it may be an excellent component of a multi long-acting drug combination.

### EXAMPLE 4

### HIV-1 Infected Subject Viral Load Suppression and PRO 140 SC Monotherapy at 350 Mg, 525 Mg, Or 700 Mg

Provided below is a study design for a multicenter study to assess the clinical safety and treatment strategy of using PRO 140 SC as long-acting single-agent maintenance therapy for 48 weeks in virologically suppressed subjects with CCR5 -tropic HIV-1 infection.

Although PRO 140 would require either subcutaneous (SC) or intravenous (IV) administration, its favorable pharmacokinetics might allow dosing as infrequent as once weekly or bi-weekly. The ability to administer the drug infrequently under medical supervision could obviate one of the continuing challenges of close adherence to daily boosted protease inhibitor regimens that appear to be relatively unforgiving in maintenance settings when administered as the sole antiretroviral regimen. This is an open-label study of PRO 140 monotherapy as maintenance therapy for subjects previously fully suppressed on combination antiretroviral regimen. PRO 140 is a promising new antiretroviral agent that does not show any cross-resistance with drugs from other classes.

The purpose and objective of this study is to assess the clinical safety and treatment strategy of using PRO 140 SC as long-acting, single-agent maintenance therapy for the chronic suppression of CCR5 -tropic HIV-1 infection. In addition, the prognostic factors of therapeutic success of PRO 140 monotherapy will be evaluated.

The primary outcome measures will be to assess the clinical safety of PRO 140 monotherapy regimen, proportion of participants experiencing virologic failure for all subjects and within each treatment group, and to evaluate the prognostic factors of therapeutic success of PRO 140 monotherapy during the Treatment Phase.

The secondary outcome measures will be time to virologic failure for all subjects and within each treatment group, proportion of participants achieving viral re-suppression (HIV-1 RNA<50 copies/mL) after experiencing virologic failure for all subjects and within each treatment group, time to achieving viral re-suppression (HIV-1 RNA<50 copies/mL) after experiencing virologic failure for all subjects and within each treatment group, proportion of virologic failure subjects achieving viral re-suppression with re-initiation of previous baseline antiretroviral regimen for all subjects and within each treatment group, proportion of participants with viral suppression (HIV-1 RNA <50 copies/mL) at week 48 for all subjects and within each treatment group, measurement of treatment adherence to the PRO 140 monotherapy regimen, mean change in CD4 cell count, at each visit within the Treatment Phase for all subjects and within each treatment group, loss of future drug options [The first occurrence of intermediate to high level resistance to any one or more of the standard antiretroviral drugs to which the patient's virus was considered to be sensitive at trial entry (i.e. excluding drug resistance present at baseline)] and proportion of participants overall and within each treatment group experiencing emerging resistance exhibited by fold increase in maraviroc and PRO 140 FC (Fold Change in IC₅₀ and IC₉₀ relative to wild-type virus) between baseline and the time of virologic failure, as a measure of post-baseline phenotypic resistance. It is noted that, here, Virologic failure is defined as two (2) consecutive plasma HIV-1 RNA levels of≥200 copies/mL.

350 subjects may be included in this phase 2b/3 study. Here, PRO 140 is indicated for use as a single-agent maintenance therapy in virally suppressed, adult subjects with CCR5 -tropic Human Immunodeficiency Virus Type-1 (HIV-1) infection who are on antiretroviral therapy. Objectives of the study are to assess the clinical safety and treatment strategy of using PRO 140 SC 350 mg or 525 mg or 700 mg as long-acting, single-agent maintenance therapy for the chronic suppression of CCR5 -tropic HIV-1 infection. In addition, the prognostic factors of therapeutic success of PRO 140 monotherapy will be evaluated.

Primary outcome measures may relate to the assessment of the clinical safety of PRO 140 monotherapy regimen, determine the proportion of participants experiencing virologic failure for all subjects and within each treatment group, and evaluate the prognostic factors of therapeutic success of PRO 140 monotherapy during the Treatment Phase. Additional outcome measures may relate to the time to virologic failure for all subjects and within each treatment group, the proportion of participants achieving viral re-suppression (HIV-1 RNA<50 copies/mL) after experiencing virologic failure for all subjects and within each treatment group, the time to achieving viral re-suppression (HIV-1 RNA<50 copies/mL) after experiencing virologic failure for all subjects and within each treatment group, the proportion of virologic failure subjects achieving viral re-suppression with re-initiation of previous baseline antiretroviral regimen for all subjects and within each treatment group, the proportion of participants with viral suppression (HIV-1 RNA <50 copies/mL) at week 48 for all subjects and within each treatment group, measurement of treatment adherence to the PRO 140 monotherapy regimen, mean change in CD4 cell count, at each visit within the Treatment Phase for all subjects and within each treatment group, and the loss of future drug options, and the proportion of participants overall and within each treatment group experiencing emerging resistance exhibited by fold increase in maraviroc and PRO 140 FC (Fold Change in IC₅₀ and IC₉₀ relative to wild-type virus) between baseline and the time of virologic failure, as a measure of post-baseline phenotypic resistance.

Here, loss of future drug options may refer to the first occurrence of intermediate to high level resistance to any one or more of the standard antiretroviral drugs to which the patient's virus was considered to be sensitive at trial entry (i.e. excluding drug resistance present at baseline). It is also noted that, here, virologic failure is defined as two (2) consecutive plasma HIV-1 RNA levels of ≥200 copies/mL.

This study is a Phase 2b/3, multi-center, randomized, two-part, open-labeled study designed to evaluate the efficacy, safety, and tolerability of the strategy of shifting clinically stable patients receiving suppressive combination antiretroviral therapy to PRO 140 monotherapy and maintaining viral suppression for 48 weeks following study entry.

Consenting patients will be shifted from combination antiretroviral regimen to weekly PRO 140 monotherapy for 48 weeks during the Treatment Phase with the one week overlap of existing retroviral regimen and PRO 140 at the beginning of the study treatment and also one week overlap at the end of the treatment in subjects who do not experience virologic failure.

In Part 1, the first 300 eligible subjects will be randomized 1:1 to PRO 140 350 mg (Group A) or PRO 140 525 mg (Group B). Once the enrollment of 300 subjects is completed, an additional 50 subjects will be randomized 1:1 to PRO 140 525 mg (Group B) or PRO 140 700 mg (Group C).

Subjects in Group A or Group B that experience virologic failure prior to week 48 in Part 1 have option of entering Part 2 wherein they receive higher dose of PRO 140 for remainder of treatment phase or may re-initiate prior ART regimen (or an alternative regimen selected by their treating physician) at the discretion of the subject and Investigator.

Part 2 may be referred to as a “rescue arm” for Group A and Group B subject. In Part 2 for Group A, single arm, open-label treatment phase for Group A subjects may elect to receive PRO 140 525 mg SC after experiencing virologic failure on 350 mg SC/weekly dose. In Part 2 for Group B: single arm, open-label treatment phase for Group B subjects may elect to receive PRO 140 700 mg SC after experiencing virologic failure on 525 mg SC/weekly dose. It is noted that all ongoing subjects assigned to Group A receiving PRO 140 350 mg SC weekly or assigned to Group B receiving PRO 140 525 mg SC weekly have the option of participating in Part 2 should virologic failure occur.

The study will have three phases: Screening Phase, Treatment Phase and Follow-up Phase.

A Screening Phase of up to 6 weeks is designed to determine whether subjects are eligible to proceed to the Treatment Phase of the study. This phase consists of a series of screening assessments designed to determine eligibility. A written informed consent from the subject will be obtained by the Investigator or suitably qualified individual before the performance of any protocol-specific procedure. Subjects will continue to take their existing antiretroviral regimen during the Screening Phase.

A Treatment Phase of up to 48 weeks ±allowed windows begins with an evaluation of results of laboratory samples collected at the Screening Visit. Subjects who meet all eligibility criteria, as per data gathered from Screening Visit are to be treated. All subjects who fail to meet eligibility criteria will be considered screen failures and exit the study without further evaluation. The first Treatment Visit (T1) will take place within 6 weeks of the Screening Visit, with weekly visits (±3 days) thereafter. Subjects will continue their existing antiretroviral regimen for up to one week after receiving initial dosing of PRO 140. The study treatment (PRO 140 350 mg or 525 mg SC or 700 mg SC injections) will be administered by a qualified medical professional (MD, DO, PA, LPN, LVN, NP, RN or CMA if permitted by state law) at clinic site or home visit or self-administered by subjects, for the duration of 48 weeks in the Treatment Phase as shown in Table 0-1.

TABLE 1-2
Randomized, two-arm, open-label treatment phase [PRO 140
350 mg or 525 mg or 700 mg]

| | Dosage | IP | Dosing Frequency and | Route of |
|---|---|---|---|---|
| Study Drug | Form | concentration | Amount | Administration |
| GROUP A | | | | |
| PRO 140 | Parenteral | 175 mg/mL | 2 injections of PRO 140 (2 × | SC injection |
| 350 mg | solution | | 1 mL/inj.) for 48 weeks | |
| GROUP B | | | | |
| PRO 140 | Parenteral | 175 mg/mL | 2 injections of PRO 140 (2 × | SC injection |
| 525 mg | solution | | 1.5 mL/inj.) for 48 weeks | |
| GROUP C | | | | |
| PRO 140 | Parenteral | 175 mg/mL | 2 injections of PRO 140 (2 × | SC injection |
| 700 mg | solution | | 2 mL/inj.) for 48 weeks | |

Group A or Group B subjects that do not elect to participate in Part 2 of Treatment Phase and all Group C subjects who experience virologic failure (defined as two consecutive HIV-1 RNA levels of >200 copies/mL) at any time during the Treatment Phase will undergo the Virologic Failure (VF) Visit assessments and then exit the Treatment Phase to enter the Follow-up Phase of the study.

Subjects who do not experience virologic failure will enter the Follow-up Phase of the study at the end of 48-week Treatment Phase.

All study subjects will re-initiate their previous antiretroviral regimen or an alternative regimen selected by their treating physician: one week prior to the end of 48-week Treatment Phase, or during the Treatment Phase, if virologic failure occurs or have met any other criteria for discontinuation of study treatment.

Efficacy assessments will include viral load measurement and CD4 cell counts at every alternate week during the first 16 weeks of Treatment Phase and once every four weeks during the remaining 32 weeks of Treatment Phase. Safety assessments will consist of determining and recording all adverse events (AEs) and severe adverse events (SAEs); laboratory evaluation of hematology, blood chemistry, and urine analysis; periodic measurement of vital signs; and the performance of physical examinations, as detailed in the schedule of procedures and assessments of the protocol.

The Follow-up Phase duration based on whether or not subject has experienced virologic failure during the Treatment Phase. Group A or Group B subjects not participating in Part 2 and Group C subjects who experience virologic failure during the Treatment Phase will be assessed every 4 weeks until the viral suppression is achieved (i.e., plasma HIV-1 RNA levels decline to <50 copies/mL). Additionally, virologic failure subjects will return to clinic for long-term follow-up at 6 months and at one year from the time of the Virologic Failure (VF) Visit. Subjects who do not experience virologic failure and complete Treatment Visit 48 (T48), will be assessed every 2 weeks for total of 4 weeks.

The duration of treatment may include a screening phase of up to 6 weeks, a treatment phase of 48 weeks ±allowed windows (up to 48 treatments every week (±3 days)), a follow-up phase wherein if a) virologic failure, until viral suppression is achieved and subjects who experience virologic failure will return to clinic for long-term follow-up at 6 months and at one year from the time of the Virologic Failure Visit, or if b) no virologic failure, 4 weeks. A total study duration may be 58 weeks does not include additional long-term follow-up time for virologic failure subjects.

Inclusion Criteria provides that potential subjects are required to meet all of the following criteria for enrollment into the study: (1) Males and females, age ≥18 years; (2) Receiving combination antiretroviral therapy for last 24 weeks; (3) No change in antiretroviral regimen within last 4 weeks prior to Screening Visit and in-between Screening Visit and First Treatment Visit; (4) Subject has two or more potential alternative approved antiretroviral drug options to consider; (5) Documented Exclusive CCR5 -tropic virus at Screening Visit as determined by Trofile™ DNA Assay; (6) Plasma HIV-1 RNA<50 copies/mL at Screening Visit as determined by Human Immunodeficiency Virus 1 (HIV-1) Quantitative, RNA (Taqman® Real-Time PCR); (7) No documented detectable viral loads (HIV-1 RNA >50 copies/mL) within the last 24 weeks prior to Screening Visit (A patient who has had one VL “blip” to <200 copies/mL in the 24 weeks prior to screening may be included, provided that the plasma HIV-1 RNA level that immediately preceded the blip and VL test that immediately followed the blip was <50 copies/mL); (8) CD4 cell count of >200 cells/mm³ since initiation of anti-retroviral therapy; (9) CD4 cell count of >350 cells/mm³ in preceding 24 weeks and at Screening Visit; (10) Laboratory values at Screening of: a. Absolute neutrophil count (ANC) greater than or equal to 750/mm³, b. Hemoglobin (Hb) greater than or equal to10.5 μm/dL (male) or greater than or equal to 9.5 μm/dL (female), c. Platelets great than or equal to 75,000 /mm³, d. Serum alanine transaminase (SGPT/ALT) less than 5× upper limit of normal (ULN), e. Serum aspartate transaminase (SGOT/AST) less than 5× ULN, f Bilirubin (total) less than 2.5× ULN unless Gilbert's disease is present or subject is receiving atazanavir in the absence of other evidence of significant liver disease, g. Creatinine less than or equal to 1.5× ULN; (11) Clinically normal resting 12-lead ECG at Screening Visit or, if abnormal, considered not clinically significant by the Principal Investigator; (12) Both male and female patients and their partners of childbearing potential must agree to use 2 medically accepted methods of contraception (e.g., barrier contraceptives [male condom, female condom, or diaphragm with a spermicidal gel], hormonal contraceptives [implants, injectables, combination oral contraceptives, transdermal patches, or contraceptive rings], and intrauterine devices) during the course of the study (excluding women who are not of childbearing potential and men who have been sterilized). Females of childbearing potential must have a negative serum pregnancy test at Screening visit and negative urine pregnancy test prior to receiving the first dose of study drug; (13) Willing and able to participate in all aspects of the study, including use of SC medication, completion of subjective evaluations, attendance at scheduled clinic visits, and compliance with all protocol requirements as evidenced by providing written informed consent. Note: Subjects diagnosed with either substance dependence or substance abuse or any history of a concomitant condition (e.g., medical, psychologic, or psychiatric) may be enrolled if in the opinion of site investigator these circumstances would not interfere with the subject's successful completion of the study requirements.

Exclusion criteria. Potential subjects meeting any of the following criteria will be excluded from enrollment: (1) CXCR4-tropic virus or dual/mixed tropic (R5×4) virus determined by the Trofile™ DNA Assay at the Screening Visit; (2) Hepatitis B infection as manifest by the presence of Hepatitis B surface antigen (HBsAg); (3) Any active infection or malignancy requiring acute therapy (with the exception of local cutaneous Kaposi's sarcoma); (4) Laboratory test values greater than or equal to grade 4 DAIDS laboratory abnormality; (5) Females who are pregnant, lactating, or breastfeeding, or who plan to become pregnant during the study; (6) Unexplained fever or clinically significant illness within 1 week prior to the first study dose; (7) Any vaccination within 2 weeks prior to the first study dose; (8) Subjects who have failed on a maraviroc containing regimen; (9) Subjects weighing <35kg; (10) History of anaphylaxis to any oral or parenteral drugs; (11) History of Bleeding Disorder or patients on anti-coagulant therapy (except aspirin) (Note: Subjects with well-controlled bleeding disorder while on stable anti-coagulant therapy dose with documented stable INRs can be enrolled as per discretion of the Investigator); (12) Participation in an experimental drug trial(s) within 30 days of the Screening Visit; (13) Any known allergy or antibodies to the study drug or excipients; (14) Treatment with any of the following: a. Radiation or cytotoxic chemotherapy with 30 days prior to the screening visit, b. Immunosuppressants within 60 days prior to the screening visit, c. Immunomodulating agents (e.g., interleukins, interferons), hydroxyurea, or foscarnet within 60 days prior to the screening visit, d. Oral or parenteral corticosteroids within 30 days prior to the Screening Visit. Subjects on chronic steroid therapy greater than 5 mg/day will be excluded with the following exception: Subjects on inhaled, nasal, or topical steroids will not be excluded; and (15) Any other clinical condition that, in the Investigator's judgment, would potentially compromise study compliance or the ability to evaluate safety/efficacy.

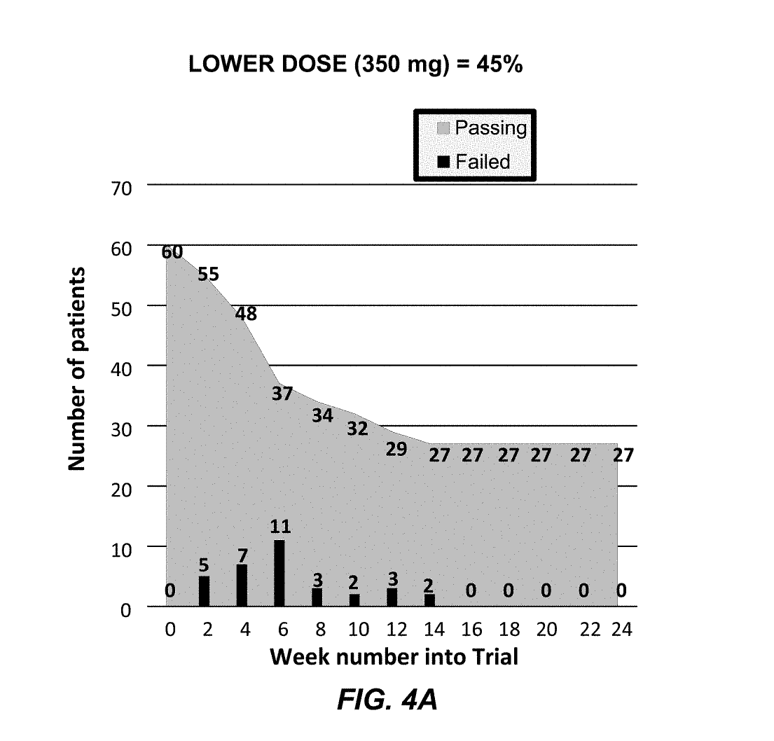

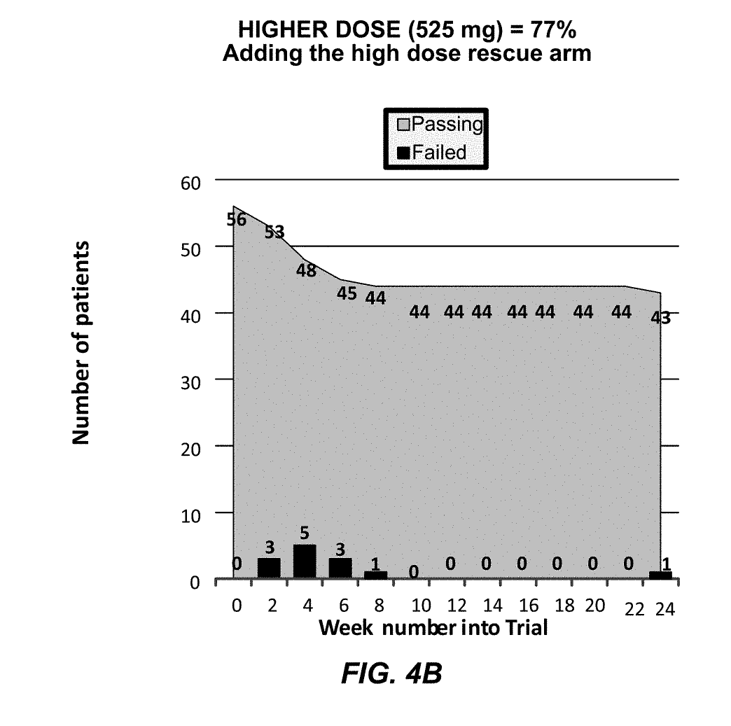

Interim results for this study are provided in FIG. 4A and FIG. 4B.

FIG. 4A provides data for 60 subjects receiving a lower dose of 350 mg weekly over 24 weeks. The graph provides that, of the 60 subjects, 27 subjects were “passing” after 24 weeks, and 33 subjects had “failed.” Thus, of the 60 subjects receiving a lower dose of 350 mg weekly over 24 weeks, about 45% were successfully passing. Interestingly, FIG. 4A also shows that none of the 33 subjects that failed did so 14 weeks into the study. That is, five (5) subjects failed at week 2, seven (7) subjects failed at week 4, eleven (11) subjects failed at week 6, three (3) subjects failed at week 8, two (2) subjects failed at week 10, three (3) subjects failed at week 12, and two (2) subjects failed at week 14. No subjects failed after 14 weeks. That is, for those twenty-seven (27) patients that were passing at 24 weeks, 100% were identified as responders by week 14.

FIG. 4B provides data for 56 subjects receiving a higher dose of 525 mg weekly over 24 weeks. The graph provides that, of the 56 subjects, 43 subjects were “passing” after 24 weeks, and 13 subjects had “failed.” Thus, of the 56 subjects receiving a higher dose of 525 mg weekly over 24 weeks, about 77% were successfully passing. Relative to the data provided in FIG. 4A, it appears that increasing the dose amount from 350 mg to 525 mg also increased the success rate at 24 weeks from about 45% to about 77%. This is an percent increase of 22%. Interestingly, FIG. 4B also shows that 12 of the 13 subjects that failed did so by 8 weeks into the study. That is, three (3) subjects failed at week 2, five (5) subjects failed at week 4, three (3) subjects failed at week 6, and one (1) subject failed at week 8. One additional subject failed at 24 weeks. For those forty-three (43) patients that were passing at 24 weeks, 92% were identified as responders by week 8. Relative to the data provided in FIG. 4A, it appears that for the majority of patients, increasing the dose amount from 350 mg to 525 mg also decreased the period of time needed to identify patients as responders from 14 weeks (100% of responders identified) to about 8 weeks (92% of responders identified). This is a time period decrease of six (6) weeks, or a percent reduction of about 43% (6 weeks/14 weeks =42.85%).

Thus, it appears that increasing the dose from 350 mg to 525 mg improves the response rate, i.e., more subjects are responders at 525 mg than at 350 mg. Second, it appears that increasing the dose from 350 mg to 525 mg shortens the time frame in which determination of which subjects are most likely to respond positively to monotherapy may be made, i.e., from about 14 weeks (350 mg) to about 8 weeks (525 mg).

The inventor further expects that these trends towards increasing the percentage of responders and shortening the time for making determinations as to which subjects will respond to monotherapy will be further enhanced for those subjects receiving 700 mg doses. For example, it is contemplated that increasing the dose from 525 mg to 700 mg may further expand the patient “pool” of responders to between about 85% and 95%, or to any of about 80%, about 81%, about 82%, about 83%, about 84%, about 85%, about 86%, about 87%, about 88%, about 89%, about 90%, about 90%, about 91%, about 92%, about 93%, about 94%, to about 95%, or greater than about 95%. For example, it is also contemplated that increasing the dose from 525 mg to 700 mg may further reduce the time frame in which determination of which subjects are most likely to respond positively to monotherapy may be made to about seven (7) weeks, about six (6) weeks, about five (5) weeks, about four (4) weeks, or less than about four (4) weeks.

In a preferred embodiment, the inventor expects that increasing the dose from 525 mg to 700 mg will increase the percentage of responders to greater than about 88% and will shorten the time for making determinations as to which subjects will respond to monotherapy to less than about six (6) weeks. In a more preferred embodiment the inventor expects that increasing the dose from 525 mg to 700 mg will increase the percentage of responders to greater than about 90% and will shorten the time for making determinations as to which subjects will respond to monotherapy to less than about six (6) weeks. In an even more preferred embodiment, the inventor expects that increasing the dose from 525 mg to 700 mg will increase the percentage of responders to greater than about 92% and will shorten the time for making determinations as to which subjects will respond to monotherapy to less than about six (6) weeks. In another preferred embodiment the inventor expects that increasing the dose from 525 mg to 700 mg will increase the percentage of responders to one of greater than about 88%, greater than about 90%, or to greater than about 92%. In another preferred embodiment the inventor expects that increasing the dose from 525 mg to 700 mg will shorten the time for making determinations as to which subjects will respond to monotherapy to less than about seven (7) weeks, less than about six (6) weeks, or less than about five (5) weeks.

All of the U.S. patents, U.S. patent application publications, U.S. patent applications, foreign patents, foreign patent applications referred to in this specification and/or listed in the Application Data Sheet are incorporated herein by reference, in their entirety. Aspects of the embodiments can be modified, if necessary to employ concepts of the various patents and applications to provide yet further embodiments. The various embodiments described above can be combined to provide further embodiments.

While specific embodiments of the invention have been illustrated and described, it will be readily appreciated that the various embodiments described above can be combined to provide further embodiments, and that various changes can be made therein without departing from the spirit and scope of the invention. These and other changes can be made to the embodiments in light of the above-detailed description.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An anti-CCR5 cell receptor single-agent dose in an amount of 700 mg.

2. (canceled)

3. The dose of claim 1, wherein the dose comprises PRO 140.

4. The dose of claim 1, wherein the dose is formulated for treatment of a CCR5 cell receptor-related condition or disease.

5. The dose of claim 1, wherein the dose is formulated for monotherapy treatment to a subject infected with HIV-1.

6. The dose of claim 1, wherein the dose is provided in a formulation concentrated to 175 mg/mL and wherein the dose is provided in two 2mL injections weekly.

7.-17. (canceled)

18. A method for treating a subject in need thereof, with an anti-CCR5 agent monotherapy, comprising:
increasing a subset size of a number of subjects suitable for treatment with an anti-CCR5 agent by administering a high dose of the anti-CCR5 agent selecting the subset of subjects suitable for treatment with the anti-CCR5 agent; and
administering the anti-CCR5 agent in an effective amount.

19. The method of claim 18, wherein the subject is infected with HIV-1.

20. The method of claim 18, wherein the anti-CCR5 agent is an antibody, or a fragment thereof.

21. The method of claim 20, wherein the antibody is PRO 140, or a fragment thereof.

22. The method of claim 21, further comprising administering PRO 140 in an amount of about 700 mg.

23. (canceled)

24. The method of claim 18, further comprising administering the anti-CCR5 agent as a monotherapy after introducing the anti-CCR5 agent as part of a combined therapy.

25. (canceled)

26. The method of claim 18, further comprising reducing the amount of time needed to determine whether a subject is suitable for treatment with the anti-CCR5 agent.

27. (canceled)

28. The method of claim 19, further comprising administering a sufficient amount of the anti-CCR5 agent to reduce the viral load to one of less than 20 copies/mL, 15 copies/mL, 10 copies/mL, 5 copies/mL, 2 copies/mL, 1 copy/mL, or to totally non-detectable levels.

29. The method of claim 28, wherein the anti-CCR5 agent is administered as part of a combined therapy before the anti-CCR5 agent is used as a monotherapy.

30. The method of claim 21, wherein PRO 140 is administered as a monotherapy for a period of time of one of at least 14 weeks, at least 24 weeks, and at least one year.

31.-32. (canceled)

33. A method for prognosis and treatment for an HIV-1-infected subject suitable for PRO 140 monotherapy comprising:
a. analyzing the subject's treatment history;
b. extracting a sample from the subject;
c. determining the subject's HIV-1 viral tropism using an in vitro assay;
d. performing an in vitro single count assay to determine the subject's viral load count;
e. applying a threshold viral load count for therapeutic efficacy that is not actually detectable or is less than or equal to 50 viral copies per mL of the subject's blood plasma to determine eligibility for PRO 140 monotherapy; and
f. administering PRO 140 monotherapy to the subject for a prolonged period of time.

34. The method of claim 33, wherein the HIV-1-infected subject comprises exclusively R5-tropic HIV-1 virus.

35. The method of claim 33, further comprising withdrawing the subject from a successful ongoing HIV-1 treatment.

36. The method of claim 33, further comprising avoiding at least one of a side effect and a toxicity associated with HAART.

37. The method of claim 33, wherein PRO 140 is administered before monotherapy, upon initiation of monotherapy, or during monotherapy, in a dose of one of about 525 mg, about 700 mg, or greater than about 700 mg.

38. The method of claim 33, further comprising administration of one or more high doses of PRO 140 in an amount effective to suppress viral load level that is not actually detectable or extremely low.

39.-56. (canceled)
